SEQUENCE LISTING (96 sequences)

SEQ ID NO: 1 - DNA, 325 bp, Artificial Sequence
  Source Nucleic acid sequence encoding LIP1-12 peptide obtained by in vitro selection from Escherichia coli bacteriophage library.
gacatccagatgacccagtctccatcctccctgtctgcatctgtaggagaccgtgtcaccatcacttgccgggcaagtaggccgattgggactaatttatattggtaccagcagaaaccagggaaagcccctaagctcctgatcgttgggggttcctttttgcaaagtggggtcccatcacgtttcagtggcagtggatctgggacagatttcactctcaccatcagcagtctgcaacctgaagattttgctacgtactactgtggtcagcgggcgcggtatcctggtacgttcggccaagggaccaaggtggaaatcaaacggg

SEQ ID NO: 2 - DNA, 325 bp, Artificial Sequence
  Source Nucleic acid sequence encoding LIP1-13 peptide obtained by in vitro selection from Escherichia coli bacteriophage library.
gacatccagatgacccagtctccatcctccctgtctgcatctgtaggagaccgtgtcaccatcacttgccgggcaagtcagggtatttggcaggagttaaagtggtaccagcagaaaccagggaaagcccctaggctcctgatctatagggggtccagtttgcaaagtggggtcccatcacgtttcagtggcagtggatctgggacagatttcactctcaccatcagcagtctgcaacctgaagattttgctacgtactactgtcaacagtcgttttatccgccttatacgttcggccaagggaccaaggtggaaatcaaacggg

SEQ ID NO: 3 - DNA, 325 bp, Artificial Sequence
  Source Nucleic acid sequence encoding LIP1-15 peptide obtained by in vitro selection from Escherichia coli bacteriophage library.
gacatccagatgacccagtctccatcctccctgtctgcatctgtaggagaccgtgtcaccatcacttgccgggcaagtcgggcgattggtacgtatttagcttggtaccagcagaaaccagggaaagcccctaagctcctgatcttgtgggggtccgtgttgcaaagtggggtcccatcacgtttcagtggcagtggatctgggacagatttcactctcaccatcagcagtctgcaacctgaagattttgctacgtactactgtgctcagttggcgcagcgtccttcgacgttcggccaagggaccaaggtggaaatcaaacggg

SEQ ID NO: 4 - DNA, 324 bp, Artificial Sequence
  Source Nucleic acid sequence encoding LIP1-17 peptide obtained by in vitro selection from Escherichia coli bacteriophage library.
gacatccagatgacccagtctccatcctccctgtctgcatctgtaggagaccgtgtcaccatcacttgccgggcaagtcagaggattggtcataagttacattggtaccagcagaaaccagggaaagcccctaagctcctgatctatcttgggtcccggttgcaaagtggggtcccatcacgtttcagtggcagtggatctgggacagatttcactctcaccatcagcagtctgcaacctgaagattttgctacgtactactgtcaacagaatgcttctaggccttatacgttcggccaagggaccaaggtggaaatcaaacgg

SEQ ID NO: 5 - DNA, 324 bp, Artificial Sequence
  Source Nucleic acid sequence encoding LIP1-19 peptide obtained by in vitro selection from Escherichia coli bacteriophage library.
gacatccagatgacccagtctccatcctccctgtctgcatctgtaggagaccgtgtcaccatcacttgccgggcaagtcagaggattgggcgtgatttagagtggtaccagcagaaaccagggaaagcccctaagctcctgatctattgggggtccattttgcaaagtggggtcccatcacgtttcagtggcagtggatctgggacagatttcactctcaccatcagcagtctgcaacctgaagattttgctacgtactactgtcaacagactatgcgttatccttttacgttcggccaagggaccaaggtggaaatcaaacgg

SEQ ID NO: 6 - DNA, 324 bp, Artificial Sequence
  Source Nucleic acid sequence encoding LIP1-21 peptide obtained by in vitro selection from Escherichia coli bacteriophage library.
gacatccagatgacccagtctccatcctccctgtctgcatctgtaggagaccgtgtcaccatcacttgccgggcaagtcagggtatttggcaggcgttacgttggtaccagcagaaaccagggagagcccctaagctcctgatctataatgggtccactttgcaaagtggggtcccatcacgcttcagtggcagtggatctgggacagatttcactctcaccatcagcagtctgcaacctgaagattttgctacgtactactgtcaacagcgtttttatcctccttatacgttcggccaagggaccaaggtggaaatcaaacgg

SEQ ID NO: 7 - DNA, 324 bp, Artificial Sequence
  Source Nucleic acid sequence encoding LIP1-22 peptide obtained by in vitro selection from Escherichia coli bacteriophage library.
gacatccagatgacccagtctccatcctccctgtctgcatctgtaggagaccgtgtcaccatcacttgccgggcaagtcagaatattatgacgcatttagcttggtatcagcagaaaccagggaaggcccctaagctcctgatctatagtcattcctatttgcaaagtggggtcccatcacgtttcagtggcagtggatctgggacagatttcactctcaccatcagcagtctgcaacctgaagattttgctacgtactactgtcaacagaagtattggcctccttttacgttcggccaagggaccaaggtggaaatcaaacgg

SEQ ID NO: 8 - DNA, 324 bp, Artificial Sequence
  Source Nucleic acid sequence encoding LIP1-23 peptide obtained by in vitro selection from Escherichia coli bacteriophage library.
gacatccagatgacccagtctccatcctccctgtctgcatctgtaggagaccgtgtcaccatcacttgccgggcaagtcaggatatttggcgtgagttaaggtggtatcagcagaaaccagggaaagcccctaagctcctgatctatcgtgcttccaatttgcaaagtggggtcccatcacgtctcagtggcagtggatctgggacagatttcactctcaccatcagcagtctgcaacctgaagattttgctacgtactactgtcaacagacgttttatcctccttatacgttcggccaagggaccaaggtggaaatcaaacgg

SEQ ID NO: 9 - DNA, 361 bp, Artificial Sequence
  Source Nucleic acid sequence encoding LIP1-26 peptide obtained by in vitro selection from Escherichia coli bacteriophage library
gaggtgcagctgttggagtctgggggaggcttggtacagcctggggggtccctgcgtctctcctgtgcagcctccggattcaccttttcgcattatacgatgagttgggtccgccaggctccagggaagggtctggagtgggtctcaagtatttcgtctactggttttaagacatactacgcagactccgtgaagggccggttcaccatctcccgcgacaattccaagaacacgctgtatctgcaaatgaacagcctgcgtgccgaggataccgcggtatattactgtgcgaaatggaatcctcataggtcgacgtattttgactactggggtcagggaaccctggtcaccgtctcgagcg

SEQ ID NO: 10 - DNA, 361 bp, Artificial Sequence
  Source Nucleic acid sequence encoding LIP1-28 peptide obtained by in vitro selection from Escherichia coli bacteriophage library.
gaggtgcagctgttggagtctgggggaggcttggtacagcctggggggtccctgcgtctctcctgtgcagcctccggattcacctttgttgctactcctatgcattgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaagggtgggatccttctttggagcggtttgactactggggtcagggaaccctggtcaccgtctcgagcg

SEQ ID NO: 11 - DNA, 358 bp, Artificial Sequence
  Source Nucleic acid sequence encoding LIP1-30 peptide obtained by in vitro selection from Escherichia coli bacteriophage library.
gaggtgcagctgttggagtctgggggaggcttggtacagcctggggggtccctgcgtctctcctgtgcagcctccggattcacctttagtgggtatagtatggcgtgggtccgccaggctccagggaagggtctggagtgggtctcacagattgggccgcttggtgttaagacatactacgcagactccgtgaagggccggttcaccatctcccgcgacaattccaagaacacgctgtatctgcaaatgaacagcctgcgtgccgaggacaccgcggtatattactgtgcgaaactgacgtcggatgctcatgattttgactactggggtcagggaaccctggtcaccgtctcgagcg

SEQ ID NO: 12 - DNA, 364 bp, Artificial Sequence
  Source Nucleic acid sequence encoding LIP1-32 peptide obtained by in vitro selection from Escherichia coli bacteriophage library.
gaggtgcagctgttggagtctgggggaggcttggtacagcctggggggtccctgcgtctctcctgtgcagcctccggattcacctttgttatgtatccgatgggttgggtccgccaggctccagggaagggtctagagtgggtctcaaagattgggcctcatggtcggctgacatactacgcagactccgtgaagggccggttcaccatctcccgcgacaattccaagaacacgctgtatctgcaaatgaacagcctgcgtgccgaggacaccgcggtatattactgtgcgaaagggaatctgtgggagatgagtcgtaggtttgactactggggtcagggaaccctggtcaccgtctcgagcg

SEQ ID NO: 13 - DNA, 358 bp, Artificial Sequence
  Source Nucleic acid sequence encoding LIP1-24 peptide obtained by in vitro selection from Escherichia coli bacteriophage library.
gaggtgcagctgttggagtctgggggaggcttggtacagcctggggggtccctgcgtctctcctgcgcagcctccggattcacctttagtagtactggtatgcattgggtccgccaggctccagggaagggtctagagtgggtctcatcgattgcggctgatggtcatactacatactacgcagactccgtgaagggccggttcaccatctcccgcgacaattccaagaacacgctgtatctgcaaatgaacagcctgcgtgccgaggacaccgcggtatattactgtgcgaaatctccgtgggtgctggataggtttgactactggggtcagggaacnctggtcaccgtctcgagcg

SEQ ID NO: 14 - DNA, 352 bp, Artificial Sequence
  Source Nucleic acid sequence encoding LIP1-25 peptide obtained by in vitro selection from Escherichia coli bacteriophage library.
gaggtgcagctgttggagtctgggggaggcttggtacagcctggggggtccctgcgtctctcctgtgcagcctccggattcacctttccggatgcggatatggtgtgggtccgccaggctccagggaagggtctagagtgggtctcatctatttctgatcatggttttaatacatactacgcagactccgtgaagggccggttcaccatctcccgcgacaattccaagaacacgctgtatctgcaaatgaacagcctgcgtgccgaggataccgcggtatattactgtgcgaaatctcctttggcgacgtttgactactggggtcagggaaccctggtcaccgtctcgagcg

SEQ ID NO: 15 - DNA, 364 bp, Artificial Sequence
  Source Nucleic acid sequence encoding LIP1-27 peptide obtained by in vitro selection from Escherichia coli bacteriophage library.
gaggtgcagctgttggagtctgggggaggcttggtacagcctggggggtccctgcgtctctcctgtgcagcctccggattcacctttgagcagactgatatgcattgggcccgccaggctccagggaagggtccagagtgggtctcatctatttcgcctttgggtgcttttacatactacgcagactccgtgaagggccggttcaccatctcccgcgacaattccaagaacacgctgtatctgcaaatgaacagcctgcgtgccgaggataccgcggtatattactgtgcgaaaagtccgctggttcgtaataatggtcagtttgactactggggtcagggaaccctggtcaccgtctcgagcg

SEQ ID NO: 16 - DNA, 358 bp, Artificial Sequence
  Source Nucleic acid sequence encoding LIP1-29 peptide obtained by in vitro selection from Escherichia coli bacteriophage library.
gaggtgcagctgttggagtctgggggaggcttggtacagcctggggggtccctgcgtctctcctgtgcagcctccggattcacctttgatcgtcgtgctatggggtgggtccgccaggctccagggaagggtctagagtgggtctcatctattgagtcggatggtactaggacatactacgcagactccgtgaagggccggttcaccatctcccgcgacaattccaagaacacgctgtatctgcaaatgaacagcctgcgtgccgaggataccgcggtatattactgtgcgaaacatcctggtagttcttatgtttttgactactggggtcagggaaccctggtcaccgtctcgagcg

SEQ ID NO: 17 - DNA, 367 bp, Artificial Sequence
  Source Nucleic acid sequence encoding LIP1-31 peptide obtained by in vitro selection from Escherichia coli bacteriophage library.
gaggtgcagctgttggagtctgggggaggcttggtacagcctggggggtccctgcgtctctcctgtgcagcctccggattcacctttgagatgtatggtatggcttgggtccgccaggctccagggaagggtctagagtgggtctcatctattagtccttcgggtcgtcatacatactacgcagactccgtgaagggccggttcaccatctcccgcgacaattccaagaacacgctgtatctgcaaatgaacagcctgcgtgccgaggataccgcggtatattactgtgcgaaacatcctcttgatctgggggagtctggtgagtttgactactggggtcagggaaccctggtcaccgtctcgagcg

SEQ ID NO: 18 - DNA, 360 bp, Artificial Sequence
  Source Nucleic acid sequence encoding LIP1-33 peptide obtained by in vitro selection from Escherichia coli bacteriophage library.
gaggtgcagctgttggagtctgggggaggcttggtacagcctggggggtccctgcgtctctcctgtgcagcctccggattcacctttccgtcgggtacgatggggtgggtccgccaggctccagggaagggtctagagtgggtctcatatattgatccgagtggttttatgacttactacgcagactccgtgaagggccggttcaccatctcccgcgacaattccaagaacacgctgtatctgcaaatgaacagcctgcgtgccgaggacaccgcggtatattactgtgcgaaaagtattgtgccgcgttcgggtacttttgactactggggtcagggaaccctggtcaccgtctcgagc

SEQ ID NO: 19 - protein, 108 aa, Artificial Sequence
  Source Amino acid sequence of LIP1-12 obtained by in vitro selection from Escherichia coli bacteriophage library.
DIQMTQSPSSLSASVGDRVTITCRASRPIGTNLYWYQQKPGKAPKLLIVGGSFLQSGVPSRFSGSGSGTDFTLTISSLQPEDFATYYCGQRARYPGTFGQGTKVEIKR

SEQ ID NO: 20 - protein, 108 aa, Artificial Sequence
  Source Amino acid sequence of LIP1-13 obtained by in vitro selection from Escherichia coli bacteriophage library.
DIQMTQSPSSLSASVGDRVTITCRASQGIWQELKWYQQKPGKAPRLLIYRGSSLQSGVPSRFSGSGSGTDFTLTISSLQPEDFATYYCQQSFYPPYTFGQGTKVEIKR

SEQ ID NO: 21 - protein, 108 aa, Artificial Sequence
  Source Amino acid sequence of LIP1-15 obtained by in vitro selection from Escherichia coli bacteriophage library.
DIQMTQSPSSLSASVGDRVTITCRASRAIGTYLAWYQQKPGKAPKLLILWGSVLQSGVPSRFSGSGSGTDFTLTISSLQPEDFATYYCAQLAQRPSTFGQGTKVEIKR

SEQ ID NO: 22 - protein, 108 aa, Artificial Sequence
  Source Amino acid sequence of LIP1-17 obtained by in vitro selection from Escherichia coli bacteriophage library.
DIQMTQSPSSLSASVGDRVTITCRASQRIGHKLHWYQQKPGKAPKLLIYLGSRLQSGVPSRFSGSGSGTDFTLTISSLQPEDFATYYCQQNASRPYTFGQGTKVEIKR

SEQ ID NO: 23 - protein, 108 aa, Artificial Sequence
  Source Amino acid sequence of LIP1-19 obtained by in vitro selection from Escherichia coli bacteriophage library.
DIQMTQSPSSLSASVGDRVTITCRASQRIGRDLEWYQQKPGKAPKLLIYWGSILQSGVPSRFSGSGSGTDFTLTISSLQPEDFATYYCQQTMRYPFTFGQGTKVEIKR

SEQ ID NO: 24 - protein, 108 aa, Artificial Sequence
  Source Amino acid sequence of LIP1-21 obtained by in vitro selection from Escherichia coli bacteriophage library.
DIQMTQSPSSLSASVGDRVTITCRASQGIWQALRWYQQKPGRAPKLLIYNGSTLQSGVPSRFSGSGSGTDFTLTISSLQPEDFATYYCQQRFYPPYTFGQGTKVEIKR

SEQ ID NO: 25 - protein, 108 aa, Artificial Sequence
  Source Amino acid sequence of LIP1-22 obtained by in vitro selection from Escherichia coli bacteriophage library.
DIQMTQSPSSLSASVGDRVTITCRASQNIMTHLAWYQQKPGKAPKLLIYSHSYLQSGVPSRFSGSGSGTDFTLTISSLQPEDFATYYCQQKYWPPFTFGQGTKVEIKR

SEQ ID NO: 26 - protein, 108 aa, Artificial Sequence
  Source Amino acid sequence of LIP1-23 obtained by in vitro selection from Escherichia coli bacteriophage library.
DIQMTQSPSSLSASVGDRVTITCRASQDIWRELRWYQQKPGKAPKLLIYRASNLQSGVPSRLSGSGSGTDFTLTISSLQPEDFATYYCQQTFYPPYTFGQGTKVEIKR

SEQ ID NO: 27 - protein, 120 aa, Artificial Sequence
  Source Amino acid sequence of LIP1-26 obtained by in vitro selection from Escherichia coli bacteriophage library.
EVQLLESGGGLVQPGGSLRLSCAASGFTFSHYTMSWVRQAPGKGLEWVSSISSTGFKTYYADSVKGRFTISRDNSKNTLYLQMNSLRAEDTAVYYCAKWNPHRSTYFDYWGQGTLVTVSS

SEQ ID NO: 28 - protein, 120 aa, Artificial Sequence
  Source Amino acid sequence of LIP1-28 obtained by in vitro selection from Escherichia coli bacteriophage library.
EVQLLESGGGLVQPGGSLRLSCAASGFTFVATPMHWVRQAPGKGLEWVSAISGSGGSTYYADSVKGRFTISRDNSKNTLYLQMNSLRAEDTAVYYCAKGWDPSLERFDYWGQGTLVTVSS

SEQ ID NO: 29 - protein, 119 aa, Artificial Sequence
  Source Amino acid sequence of LIP1-30 obtained by in vitro selection from Escherichia coli bacteriophage library.
EVQLLESGGGLVQPGGSLRLSCAASGFTFSGYSMAWVRQAPGKGLEWVSQIGPLGVKTYYADSVKGRFTISRDNSKNTLYLQMNSLRAEDTAVYYCAKLTSDAHDFDYWGQGTLVTVSS

SEQ ID NO: 30 - protein, 121 aa, Artificial Sequence
  Source Amino acid sequence of LIP1-32 obtained by in vitro selection from Escherichia coli bacteriophage library.
EVQLLESGGGLVQPGGSLRLSCAASGFTFVMYPMGWVRQAPGKGLEWVSKIGPHGRLTYYADSVKGRFTISRDNSKNTLYLQMNSLRAEDTAVYYCAKGNLWEMSRRFDYWGQGTLVTVSS

SEQ ID NO: 31 - protein, 119 aa, Artificial Sequence
  Source Amino acid sequence of LIP1-24 obtained by in vitro selection from Escherichia coli bacteriophage library.
EVQLLESGGGLVQPGGSLRLSCAASGFTFSSTGMHWVRQAPGKGLEWVSSIAADGHTTYYADSVKGRFTISRDNSKNTLYLQMNSLRAEDTAVYYCAKSPWVLDRFDYWGQGXLVTVSS

SEQ ID NO: 32 - protein, 117 aa, Artificial Sequence
  Source Amino acid sequence of LIP1-25 obtained by in vitro selection from Escherichia coli bacteriophage library.
EVQLLESGGGLVQPGGSLRLSCAASGFTFPDADMVWVRQAPGKGLEWVSSISDHGFNTYYADSVKGRFTISRDNSKNTLYLQMNSLRAEDTAVYYCAKSPLATFDYWGQGTLVTVSS

SEQ ID NO: 33 - protein, 121 aa, Artificial Sequence
  Source Amino acid sequence of LIP1-27 obtained by in vitro selection from Escherichia coli bacteriophage library.
EVQLLESGGGLVQPGGSLRLSCAASGFTFEQTDMHWARQAPGKGPEWVSSISPLGAFTYYADSVKGRFTISRDNSKNTLYLQMNSLRAEDTAVYYCAKSPLVRNNGQFDYWGQGTLVTVSS

SEQ ID NO: 34 - protein, 119 aa, Artificial Sequence
  Source Amino acid sequence of LIP1-29 obtained by in vitro selection from Escherichia coli bacteriophage library.
EVQLLESGGGLVQPGGSLRLSCAASGFTFDRRAMGWVRQAPGKGLEWVSSIESDGTRTYYADSVKGRFTISRDNSKNTLYLQMNSLRAEDTAVYYCAKHPGSSYVFDYWGQGTLVTVSS

SEQ ID NO: 35 - protein, 122 aa, Artificial Sequence
  Source Amino acid sequence of LIP1-31 obtained by in vitro selection from Escherichia coli bacteriophage library.
EVQLLESGGGLVQPGGSLRLSCAASGFTFEMYGMAWVRQAPGKGLEWVSSISPSGRHTYYADSVKGRFTISRDNSKNTLYLQMNSLRAEDTAVYYCAKHPLDLGESGEFDYWGQGTLVTVSS

SEQ ID NO: 36 - protein, 120 aa, Artificial Sequence
  Source Amino acid sequence of LIP1-33 obtained by in vitro selection from Escherichia coli bacteriophage library.
EVQLLESGGGLVQPGGSLRLSCAASGFTFPSGTMGWVRQAPGKGLEWVSYIDPSGFMTYYADSVKGRFTISRDNSKNTLYLQMNSLRAEDTAVYYCAKSIVPRSGTFDYWGQGTLVTVSS

SEQ ID NO: 37 - protein, 40 aa, Artificial Sequence
  Source C-terminal peptide from Homo sapiens DC-SIGN amino acid sequence obtained by standard in vitro molecular biology techniques.
HHHHHHHHHSGSGKKSAASCSRDEEQFLSPAPATPNPPPA

SEQ ID NO: 38 - protein, 15 aa, Artificial Sequence
  Source poly His tag obtained by standard in vitro molecular biology techniques.
GHHGHHGHHGHHGHH

SEQ ID NO: 39 - protein, 152 aa, Homo sapiens
CHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 40 - protein, 135 aa, Homo sapiens
CRHCPKDWTFFQGNCYFMSNSQRNWHNSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 41 - protein, 404 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 42 - protein, 353 aa, Homo sapiens [sequence omitted]

SEQ ID NO: 43 - protein, 11 aa, Artificial Sequence
  Source CDR identified by in silico analysis.
RASRPIGTNLY

SEQ ID NO: 44 - protein, 11 aa, Artificial Sequence
  Source CDR identified by in silico analysis.
RASQGIWQELK

SEQ ID NO: 45 - protein, 11 aa, Artificial Sequence
  Source CDR identified by in silico analysis.
RASRAIGTYLA

SEQ ID NO: 46 - protein, 11 aa, Artificial Sequence
  Source CDR identified by in silico analysis.
RASQRIGHKLH

SEQ ID NO: 47 - protein, 11 aa, Artificial Sequence
  Source CDR identified by in silico analysis.
RASQRIGRDLE

SEQ ID NO: 48 - protein, 11 aa, Artificial Sequence
  Source CDR identified by in silico analysis.
RASQGIWQALR

SEQ ID NO: 49 - protein, 11 aa, Artificial Sequence
  Source CDR identified by in silico analysis.
RASQNIMTHLA

SEQ ID NO: 50 - protein, 11 aa, Artificial Sequence
  Source CDR identified by in silico analysis.
RASQDIWRELR

SEQ ID NO: 51 - protein, 5 aa, Artificial Sequence
  Source CDR identified by in silico analysis.
STGMH

SEQ ID NO: 52 - protein, 5 aa, Artificial Sequence
  Source CDR identified by in silico analysis.
DADMV

SEQ ID NO: 53 - protein, 5 aa, Artificial Sequence
  Source CDR identified by in silico analysis.
HYTMS

SEQ ID NO: 54 - protein, 5 aa, Artificial Sequence
  Source CDR identified by in silico analysis.
QTDMH

SEQ ID NO: 55 - protein, 5 aa, Artificial Sequence
  Source CDR identified by in silico analysis.
ATPMH

SEQ ID NO: 56 - protein, 5 aa, Artificial Sequence
  Source CDR identified by in silico analysis.
RRAMG

SEQ ID NO: 57 - protein, 5 aa, Artificial Sequence
  Source CDR identified by in silico analysis.
GYSMA

SEQ ID NO: 58 - protein, 5 aa, Artificial Sequence
  Source CDR identified by in silico analysis.
MYGMA

SEQ ID NO: 59 - protein, 5 aa, Artificial Sequence
  Source CDR identified by in silico analysis.
MYPMG

SEQ ID NO: 60 - protein, 5 aa, Artificial Sequence
  Source CDR identified by in silico analysis.
SGTMG

SEQ ID NO: 61 - protein, 7 aa, Artificial Sequence
  Source CDR identified by in silico analysis.
GGSFLQS

SEQ ID NO: 62 - protein, 7 aa, Artificial Sequence
  Source CDR identified by in silico analysis.
RGSSLQS

SEQ ID NO: 63 - protein, 7 aa, Artificial Sequence
  Source CDR identified by in silico analysis.
WGSVLQS

SEQ ID NO: 64 - protein, 7 aa, Artificial Sequence
  Source CDR identified by in silico analysis.
LGSRLQS

SEQ ID NO: 65 - protein, 7 aa, Artificial Sequence
  Source CDR identified by in silico analysis.
WGSILQS

SEQ ID NO: 66 - protein, 7 aa, Artificial Sequence
  Source CDR identified by in silico analysis.
NGSTLQS

SEQ ID NO: 67 - protein, 7 aa, Artificial Sequence
  Source CDR identified by in silico analysis.
SHSYLQS

SEQ ID NO: 68 - protein, 7 aa, Artificial Sequence
  Source CDR identified by in silico analysis.
RASNLQS

SEQ ID NO: 69 - protein, 17 aa, Artificial Sequence
  Source CDR identified by in silico analysis.
SIAADGHTTYYADSVKG

SEQ ID NO: 70 - protein, 17 aa, Artificial Sequence
  Source CDR identified by in silico analysis.
SISDHGFNTYYADSVKG

SEQ ID NO: 71 - protein, 17 aa, Artificial Sequence
  Source CDR identified by in silico analysis.
SISSTGFKTYYADSVKG

SEQ ID NO: 72 - protein, 17 aa, Artificial Sequence
  Source CDR identified by in silico analysis.
SISPLGAFTYYADSVKG

SEQ ID NO: 73 - protein, 17 aa, Artificial Sequence
  Source CDR identified by in silico analysis.
AISGSGGSTYYADSVKG

SEQ ID NO: 74 - protein, 17 aa, Artificial Sequence
  Source CDR identified by in silico analysis.
SIESDGTRTYYADSVKG

SEQ ID NO: 75 - protein, 17 aa, Artificial Sequence
  Source CDR identified by in silico analysis.
QIGPLGVKTYYADSVKG

SEQ ID NO: 76 - protein, 17 aa, Artificial Sequence
  Source CDR identified by in silico analysis.
SISPSGRHTYYADSVKG

SEQ ID NO: 77 - protein, 17 aa, Artificial Sequence
  Source CDR identified by in silico analysis.
KIGPHGRLTYYADSVKG

SEQ ID NO: 78 - protein, 17 aa, Artificial Sequence
  Source CDR identified by in silico analysis.
YIDPSGFMTYYADSVKG

SEQ ID NO: 79 - protein, 9 aa, Artificial Sequence
  Source CDR identified by in silico analysis.
GQRARYPGT

SEQ ID NO: 80 - protein, 9 aa, Artificial Sequence
  Source CDR identified by in silico analysis.
QQSFYPPYT

SEQ ID NO: 81 - protein, 9 aa, Artificial Sequence
  Source CDR identified by in silico analysis.
AQLFQRPST

SEQ ID NO: 82 - protein, 9 aa, Artificial Sequence
  Source CDR identified by in silico analysis.
QQNASRPYT

SEQ ID NO: 83 - protein, 9 aa, Artificial Sequence
  Source CDR identified by in silico analysis.
QQTMSRPFT

SEQ ID NO: 84 - protein, 9 aa, Artificial Sequence
  Source CDR identified by in silico analysis.
QQRFYPPYT

SEQ ID NO: 85 - protein, 9 aa, Artificial Sequence
  Source CDR identified by in silico analysis.
QQKYWPPFT

SEQ ID NO: 86 - protein, 9 aa, Artificial Sequence
  Source CDR identified by in silico analysis.
QQTFYPPYT

SEQ ID NO: 87 - protein, 11 aa, Artificial Sequence
  Source CDR identified by in silico analysis.
SPWVLDRFDYW

SEQ ID NO: 88 - protein, 9 aa, Artificial Sequence
  Source CDR identified by in silico analysis.
SPLATFDYW

SEQ ID NO: 89 - protein, 12 aa, Artificial Sequence
  Source CDR identified by in silico analysis.
WNPHRSTYFDYW

SEQ ID NO: 90 - protein, 13 aa, Artificial Sequence
  Source CDR identified by in silico analysis.
SPLVRNNGQFDYW

SEQ ID NO: 91 - protein, 12 aa, Artificial Sequence
  Source CDR identified by in silico analysis.
GWDPSLERFDYW

SEQ ID NO: 92 - protein, 11 aa, Artificial Sequence
  Source CDR identified by in silico analysis.
HPGSSYVFDYW

SEQ ID NO: 93 - protein, 11 aa, Artificial Sequence
  Source CDR identified by in silico analysis.
LTSDAHDFDYW

SEQ ID NO: 94 - protein, 14 aa, Artificial Sequence
  Source CDR identified by in silico analysis.
HPLDLGESGEFDYW

SEQ ID NO: 95 - protein, 13 aa, Artificial Sequence
  Source CDR identified by in silico analysis.
GNLWEMSRRFDYW

SEQ ID NO: 96 - protein, 12 aa, Artificial Sequence
  Source CDR identified by in silico analysis.
SIVPRSGTFDYW

(19) United States
(12) Patent Application Publication
(10) Pub. No.: US 2011/0257373 A1
(43) Pub. Date: Oct. 20, 2011

(54) LIGANDS THAT HAVE BINDING SPECIFICITY FOR DC-SIGN

(76) Inventor: Rudolf Maria De Wildt, Hertfordshire (GB)

(21) Appl. No.: 13/125,384

(22) PCT Filed: Oct. 19, 2009
(86) PCT No.: PCT/EP09/63655

§ 371 Date: Apr. 21, 2011

Related U.S. Application Data
(60) Provisional application No. 61/107,085, filed on Oct. 21, 2008.

(51) Int. Cl. C07K 16/28; C12P 21/02; C12N 1/00; C07H 21/00; C12N 15/63

(52) U.S. Cl. 530/389.6; 536/23.53; 435/320.1; 435/243; 435/69.6

(57) ABSTRACT

The present invention disclosure provides an anti-dendritic cell-specific ICAM-3 grabbing non-integrin (DC-SIGN; CD209) immunoglobulin single variable domain. Polypeptides, ligands and compositions comprising such anti-DC-SIGN immunoglobulin single variable domains are also described along with nucleic acids encoding such immunoglobulins and vectors and host cells for expression. The invention disclosure further relates to uses, formulations, compositions and devices comprising such DC-SIGN-binding agents.

This application is a 371 of International Application No. PCT/EP2009/063655, filed Oct. 19, 2009, which claims the benefit of U.S. Provisional Application No. 61/107,085, filed Oct. 21, 2008, which is incorporated herein in its entirety.

The present disclosure relates to agents that bind DC-SIGN. In particular, the present disclosure relates to immunoglobulin single variable domains which bind to DC-SIGN. The disclosure further relates to uses, formulations, compositions and devices comprising such DC-SIGN-binding agents.

### BACKGROUND OF THE DISCLOSURE

Dendritic cell-specific ICAM-3 grabbing non-integrin (DC-SIGN or CD209) is a type II membrane protein that is a mannose specific calcium dependent (C-type) lectin. DC-SIGN mediates interactions between dendritic cells (DCs) and T cells and shares 77% homology with the related molecule, DC-SIGNR. Both DC-SIGN and DC-SIGNR have been shown to bind HIV, hepatitis C glycoproteins, Ebola virus glycol proteins and the cellular adhesion protein ICAM-3. DC-SIGN is expressed solely on dendritic cells while DC-SIGNR is found on endothelial cells in the liver, lymph node sinuses and in endothelial cells in the placenta.

Dendritic cells (DCs) are specialized antigen-presenting cells capable of activating naïve and memory T-lymphocytes. Harnessing their properties has become the focus of immunotherapeutic strategies against disease including cancer.

Antibodies that bind DC-SIGN have been produced but there is a need for improved binding agents.

### SUMMARY OF THE DISCLOSURE

In one aspect, the disclosure provides an anti-dendritic cell-specific ICAM-3 grabbing non-integrin (DC-SIGN; CD209) immunoglobulin single variable domain.

In one embodiment, the immunoglobulin single variable domain binds to human DC-SIGN with a dissociation constant (K_{d}) of 1 to 50 μM, as determined by surface plasmon resonance.

In one aspect, the disclosure provides a polypeptide comprising an amino acid sequence that is at least 70, 75, 80, 85 or 90% identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 as shown in FIG. 4 and set out in SEQ ID NOs: 19 to 36. In one embodiment, the percent identity is at least 91, 92, 93, 94, 95, 96, 97, 98 or 99%. In one embodiment, the polypeptide is any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33. The disclosure further provides (substantially) pure any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 monomer. In one embodiment, the any one of LIP1-12, LIP1-13, LIP1-15,

LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 is at least 98, 99, 99.5% pure or 100% pure monomer. Suitably, the polypeptide binds DC-SIGN.

In one aspect, the disclosure provides a polypeptide encoded by a nucleotide sequence that is at least 60, 65, 70, 75 or 80% identical to the nucleotide sequence of any of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 as shown in FIG. 3 and set out in SEQ ID NOs: 1 to 18. In one embodiment, the percent identity is at least 70, 80, 85, 90, 91, 92, 93, 94, 95, 96, 97, 98 or 99%. Suitably, the polypeptide encoded by the nucleotide sequence binds DC-SIGN.

In one aspect, the disclosure provides an anti-dendritic cell-specific ICAM-3 grabbing non-integrin (DC-SIGN; CD209) immunoglobulin single variable domain comprising an amino acid sequence that is at least 70, 75, 80, 85 or 90% identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP 1-31, LIP1-32 or LIP1-33. In one embodiment, the percent identity is at least 91, 92, 93, 94, 95, 96, 97, 98 or 99%.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 as shown in FIG. 4 and set out in SEQ ID NOs: 19 to 36.

In one aspect, the disclosure provides an anti-DC-SIGN immunoglobulin single variable domain comprising an amino acid sequence that is identical to the amino acid sequence of LIP1-29. In another aspect, the disclosure provides an anti-DC-SIGN immunoglobulin single variable domain comprising an amino acid sequence that is identical to the amino acid sequence of LIP1-30.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP 1-32 or LIP1-33 or differs from the amino acid sequence of any one of LIP 1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 at no more than 25 amino acid positions and has a CDR1 sequence that is at least 50% identical to the CDR1 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 . In one embodiment, the difference is no more than 24, 23, 22, 21, 20, 19, 18, 17, 16, 15, 14, 13, 12, 11, 10, 9, 8, 7, 6, 5, 4, 3, 2 or 1 amino acid positions. In one embodiment, the CDR sequence identity is at least 55, 60, 65, 70, 75, 80, 85, 90, 95, 96, 97, 98 or 99%.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 or differs from the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 at no more than 25 amino acid positions and has a CDR2 sequence that is at least 50% identical to the CDR2 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33. In one embodiment, the difference is no more than 24, 23, 22, 21, 20, 19, 18, 17, 16, 15, 14, 13, 12, 11, 10, 9, 8, 7, 6, 5, 4, 3, 2 or 1 amino acid position. In one embodiment, the CDR sequence identity is at least 55, 60, 65, 70, 75, 80, 85, 90, 95, 96, 97, 98 or 99%.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 or differs from the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 at no more than 25 amino acid positions and has a CDR3 sequence that is at least 50% identical to the CDR3 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33. In one embodiment, the difference is no more than 24, 23, 22, 21, 20, 19, 18, 17, 16, 15, 14, 13, 12, 11, 10, 9, 8, 7, 6, 5, 4, 3, 2 or 1 amino acid position. In one embodiment, the CDR sequence identity is at least 55, 60, 65, 70, 75, 80, 85, 90, 95, 96, 97, 98 or 99%.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 or differs from the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 at no more than 25 amino acid positions and has a CDR1 sequence that is at least 50% identical to the CDR1 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 and has a CDR2 sequence that is at least 50% identical to the CDR2 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33. In one embodiment, the difference is no more than 24, 23, 22, 21, 20, 19, 18, 17, 16, 15, 14, 13, 12, 11, 10, 9, 8, 7, 6, 5, 4, 3, 2 or 1 amino acid position. In one embodiment, the CDR sequence identity is at least 55, 60, 65, 70, 75, 80, 85, 90, 95, 96, 97, 98 or 99%.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 or differs from the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 at no more than 25 amino acid positions and has a CDR1 sequence that is at least 50% identical to the CDR1 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 and has a CDR3 sequence that is at least 50% identical to the CDR3 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33. In one embodiment, the difference is no more than 24, 23, 22, 21, 20, 19, 18, 17, 16, 15, 14, 13, 12, 11, 10, 9, 8, 7, 6, 5, 4, 3, 2 or 1 amino acid position. In one embodiment, the CDR sequence identity is at least 55, 60, 65, 70, 75, 80, 85, 90, 95, 96, 97, 98 or 99%.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 or differs from the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 at no more than 25 amino acid positions and has a CDR2 sequence that is at least 50% identical to the CDR2 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 and has a CDR3 sequence that is at least 50% identical to the CDR3 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33. In one embodiment, the difference is no more than 24, 23, 22, 21, 20, 19, 18, 17, 16, 15, 14, 13, 12, 11, 10, 9, 8, 7, 6, 5, 4, 3, 2 or 1 amino acid position. In one embodiment, the CDR sequence identity is at least 55, 60, 65, 70, 75, 80, 85, 90, 95, 96, 97, 98 or 99%.

In one aspect, the disclosure provides an anti-DC-SIGN immunoglobulin single variable domain comprising an amino acid sequence that is identical to the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 or differs from the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 at no more than 25 amino acid positions and has a CDR1 sequence that is at least 50% identical to the CDR1 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 and has a CDR2 sequence that is at least 50% identical to the CDR2 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33 and has a CDR3 sequence that is at least 50% identical to the CDR3 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33. In one embodiment, the difference is no more than 24, 23, 22, 21, 20, 19, 18, 17, 16, 15, 14, 13, 12, 11, 10, 9, 8, 7, 6, 5, 4, 3, 2 or 1 amino acid position. In one embodiment, the CDR sequence identity is at least 55, 60, 65, 70, 75, 80, 85, 90, 95, 96, 97, 98 or 99%.

In one aspect, the disclosure provides an anti-DC-SIGN immunoglobulin single variable domain comprising the CDR3 sequence from any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 or a CDR3 sequence that is at least 50% identical to the CDR3 sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33. In one embodiment, the difference is no more than 14, 13, 12, 11, 10, 9, 8, 7, 6, 5, 4, 3, 2 or 1 amino acid position. In one embodiment, the CDR sequence identity is at least 55, 60, 65, 70, 75, 80, 85, 90, 95, 96, 97, 98 or 99%. In one embodiment, an anti-DC-SIGN immunoglobulin single variable domain comprises a CDR3 sequence from any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33.

In another aspect, the disclosure provides anti-DC-SIGN immunoglobulin single variable domain comprising the sequence of CDR1, CDR2, and/or CDR3 (e.g., CDR1, CDR2, CDR3, CDR1 and 2, CDR1 and 3, CDR2 and 3 or CDR1, 2 and 3) of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32, LIP1-33.

In one embodiment, CDR sequences 1, 2 or 3 for any anti-DC-SIGN immunoglobulin single variable domains in accordance with the disclosure are as set out in FIG. 5, 6 or 8. In one embodiment of any aspect of the disclosure, the anti-DC-SIGN immunoglobulin single variable domain binds to DC-SIGN.

In one embodiment, the anti-DC-SIGN immunoglobulin single variable domain in accordance with any aspect of the disclosure binds specifically to DC-SIGN but not to DC-SIGNR.

In one embodiment, the anti-DC-SIGN immunoglobulin single variable domain in accordance with any aspect of the disclosure binds to DC-SIGN with low affinity. In one embodiment, the affinity of the anti-DC-SIGN immunoglobulin single variable domain in accordance with the disclosure for DC-SIGN is 1 μM or higher.

In another aspect, the disclosure provides a ligand that has binding specificity for DC-SIGN and inhibits the binding of an anti-DC-SIGN immunoglobulin single variable domain having the amino acid sequence of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 to DC-SIGN.

In a further aspect of the disclosure there is provided an anti-DC-SIGN immunoglobulin single variable domain which immunoglobulin single variable domain has the binding specificity of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 i.e. any of SEQ ID NOS: 19 to 36.

In one embodiment, the amino acid sequence of the polypeptide or anti-DC-SIGN immunoglobulin single variable domain in accordance with the disclosure may comprise additional amino acids at the N or C terminal end to facilitate expression and/or use of the polypeptide or single variable domain. In one embodiment, the polypeptide or anti-DC-SIGN immunoglobulin single variable domain may comprise amino acids ST N-terminal to the amino acid sequence as set out in any of SEQ ID NOS: 19 to 36. In another embodiment, the polypeptide or anti-DC-SIGN immunoglobulin single variable domain may comprise a tag sequence such as a polyhistidine tag (His-tag). In one embodiment polypeptide or anti-DC-SIGN immunoglobulin single variable domain may comprise a His-tag at the C-terminal.

In one aspect, the disclosure provides a polypeptide encoded by a nucleotide sequence that is at least 80% identical to the nucleotide sequence of any of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33 as shown in FIG. 3 and set out in SEQ ID NOs: 1 to 18 and wherein the polypeptide comprises an amino acid sequence that is at least 90% identical to the amino acid sequence of any LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33.

In one embodiment, the percent identity of the nucleotide sequence is at least 80, 85, 90, 91, 92, 93, 94, 95, 96, 97, 98 or 99%. In one embodiment, the percent identity of the amino acid sequence is at least 91, 92, 93, 94, 95, 96, 97, 98 or 99% or 100%. For example, the nucleotide sequence may be a codon-optimised version of the nucleotide sequence of any LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33. Codon optimization of sequences is known in the art. In one embodiment, the nucleotide sequence is optimized for expression in a bacterial (e.g., E. coli or Pseudomonas, e.g. P fluorescens), mammalian (e.g.e.g., CHO) or yeast host cell (e.g. Picchia or Saccharomyces, e.g. P. pastoris or S. cerevisiae).

In one aspect, the disclosure provides a fusion protein comprising the polypeptide of the disclosure.

In one aspect, the disclosure provides an isolated or recombinant nucleic acid encoding a polypeptide comprising an immunoglobulin single variable domain in accordance with any aspect of the disclosure. In one aspect, the disclosure provides a vector comprising the nucleic acid. In one embodiment, the vector is an expression vector comprising a leader sequence such as a GAS leader sequence (as described, for example, in WO 2005/093074) to ensure expression in the cell supernatant. In one aspect, the disclosure provides a host cell comprising the nucleic acid or the vector. In one embodiment, the host cell is E.Coli. Suitable strains of E.Coli will be familiar to those skilled in the art and include, for example, HB2151 cells or BL21 cells. In one aspect, the disclosure provides a method of producing a polypeptide comprising an immunoglobulin single variable domain, the method comprising maintaining the host cell under conditions suitable for expression of said nucleic acid or vector, whereby a polypeptide comprising an immunoglobulin single variable domain is produced. The method may further comprise purification of the polypeptide. The method may further comprise isolating the polypeptide, variable domain or binding agent and optionally producing a variant, e.g. a mutated variant, having an improved affinity and/or ND50 (50% neutralizing dose) than the isolated polypeptide, variable domain or binding agent. Techniques for improving binding affinity of immunoglobulin single variable domains are known in the art, e.g. techniques for affinity maturation.

In one aspect, the disclosure provides a pharmaceutical composition comprising an immunoglobulin single variable domain, polypeptide or binding agent in accordance with any aspect of the disclosure, and a pharmaceutically acceptable carrier, excipient or diluent.

In one embodiment, the immunoglobulin single variable domain in accordance with the disclosure comprises an antibody constant domain, for example, an antibody Fc, optionally wherein the N-terminus of the Fc is linked (optionally directly linked) to the C-terminus of the variable domain.

The polypeptide or variable domain of the disclosure can be isolated and/or recombinant.

In one aspect, there is provided a DC-SIGN binding agent comprising a polypeptide or variable domain in accordance with any aspect of the disclosure. Suitably a “DC-SIGN binding agent” is an agent which binds to DC-SIGN and which comprises an anti-DC-SIGN immunoglobulin single variable domain in accordance with the disclosure. In one embodiment, the binding agent is an anti-DC-SIGN immunoglobulin single variable domain in a carrier. Suitably the carrier may be a lipid-based carrier such as a membrane vesicle or liposome. In one embodiment, the anti-DC-SIGN immunoglobulin single variable domain is carried by a carrier or is on a carrier.

In one embodiment, the composition comprising an anti-DC-SIGN immunoglobulin single variable domain in a carrier confers an increased half-life on the anti-DC-SIGN immunoglobulin single variable domain.

In another aspect, an increased half-life can be conferred on the anti-DC-SIGN immunoglobulin single variable domain through fusion with another moiety.

There is further described herein a diagnostic kit to determine whether DC-SIGN is present in a sample or how much DC-SIGN is present in a sample, comprising a polypeptide, immunoglobulin variable domain (dAb) or binding agent of the disclosure and instructions for use (e.g., to determine the presence and/or quantity of DC-SIGN in the sample). In some embodiments, the kit further comprises one or more ancillary reagents, such as a suitable buffer or suitable detecting reagent (e.g., a detectably labeled antibody or antigen-binding fragment thereof that binds the polypeptide or dAb of the disclosure or a moiety associated or conjugated thereto).

The disclosure also relates to a device comprising a solid surface on which a polypeptide, antagonist or dAb of the disclosure is immobilized such that the immobilized polypeptide or dAb binds DC-SIGN. Any suitable solid surfaces on which an antibody or antigen-binding fragment thereof can be immobilized can be used, for example, glass, plastics, carbohydrates (e.g., agarose beads). If desired the support can contain or be modified to contain desired functional groups to facilitate immobilization. The device, and or support, can have any suitable shape, for example, a sheet, rod, strip, plate, slide, bead, pellet, disk, gel, tube, sphere, chip, plate or dish, and the like. In some embodiments, the device is a dipstick. In one embodiment, such a device may be used for purification or isolation of dendritic cells.

In another aspect, there is provided a composition comprising an anti-DC-SIGN single variable domain in accordance with the disclosure for use as a medicament. In one embodiment, the anti-DC-SIGN single variable domain may be utilized in the delivery of compounds to dendritic cells through its specific binding to DC-SIGN. One suitable use for such delivery can be in generating an immune response. In particular, an antitumour response may be generated. Accordingly, the disclosure provides a composition for use in the treatment of cancer, for example melanoma. In another aspect, the disclosure provides a composition for use in the treatment of infections where the infectious agent enters cells through binding to DC-SIGN. Examples of such infections include viral infections such as HIV, Hepatitis C and Ebola virus. Accordingly, the disclosure further provides a composition comprising an anti-DC-SIGN single variable domain in accordance with the disclosure for use in treating HIV, Hepatitis C or Ebola. The disclosure also provides use of a composition comprising an anti-DC-SIGN single variable domain in accordance with the disclosure in the manufacture of a medicament for use in the treatment of infections. The disclosure further provides a method of treating cancer or infection comprising administering composition comprising an anti-DC-SIGN single variable domain in accordance with the disclosure.

### BRIEF DESCRIPTION OF THE DRAWINGS

FIG. 1 shows LIP1 phage particles binding to DC-SIGN coated plates. Phage particles from individual LIP1 clones were prepared, and serial dilutions of phage particles were tested in ELISA. The ELISA wells were coated overnight at 4° C. with DC SIGN (1-10 μg/ml in PBS or 0.1 M NaHCO₃ buffer, pH 9.6). After blocking the wells with PBS containing 2% skimmed-milk powder (PBSM), phage was incubated in PBSM for 1 hr. After washing with PBS, bound phage were detected using a conjugate of horseradish peroxidase with an anti-M13 monoclonal antibody (Amersham) using 3,3′,5,5′-tetramethylbenzidine as substrate. HEL4 (Jespers et al., J. Mol. Biol. (2004) 337, 893-903) and VKdummy are non-binding negative controls.

FIG. 2 shows LIP1-33 phage binding to DC-SIGN peptide and DC SIGN. Phage particles from LIP1-33 were prepared, and serial dilutions of phage particles were tested in ELISA. The ELISA wells were coated overnight at 4° C. with DC SIGNR, neutravidin, DC SIGN or DC SIGN peptide (1-10 μg/ml in PBS or 0.1 M NaHCO₃ buffer, pH 9.6). After blocking the wells with PBS containing 2% skimmed-milk powder (PBSM), phage was incubated in PBSM for 1 hr. After washing with PBS, bound phage were detected using a conjugate of horseradish peroxidase with an anti-M13 monoclonal antibody (Amersham) using 3,3′,5,5′-tetramethylbenzidine as substrate. HEL4 and VKdummy are non-binding negative controls.

FIG. 3 shows nucleotide sequences from LIP1 VH and VK dAbs. “˜” indicates spaces which have been introduced into the sequences presented in FIG. 3 to allow for sequence alignment of dAb sequences.

FIG. 4 shows amino acid sequences from LIP1 VH and VK dAbs. “˜” indicates spaces which have been introduced into the sequences presented in FIG. 4 to allow for sequence alignment of dAb sequences.

FIG. 5 shows amino acid alignment of sequences from LIP1 VK dAbs. Amino acid numbering is according to Kabat. Where a dot (“.”) is indicated in the alignment, the dAb sequence is identical to the first listed reference dAb. Variant amino acids are indicated by single letter amino acid code. The sequences represented herein are also shown in full in FIG. 4. Sequences highlighted in bold and underlined represent CDR1, CDR2 and CDR3 sequences, consecutively. “-” indicates spaces which have been introduced into the sequences presented in FIG. 5 to allow for sequence alignment of all dAb sequences.

FIG. 6 shows amino acid alignment of sequences from LIP1 VH dAbs. Amino acid numbering is according to Kabat. Where a dot (“.”) is indicated in the alignment, the dAb sequence is identical to the first listed reference dAb. Variant amino acids are indicated by single letter amino acid code. The sequences represented herein are also shown in full in FIG. 4. Sequences highlighted in bold represent CDR1, CDR2 and CDR3 sequences, consecutively. “-” indicates spaces which have been introduced into the sequences presented in FIG. 5 to allow for sequence alignment of all dAb sequences.

FIG. 7 shows alignment of human DC-SIGN vs. DC-SIGNR. Identical amino acids as well as conservative substitutions are indicated. Homology for full length protein (A) is 69% and for carbohydrate recognition domain (CRD) (B) is 71%. The amino acid sequence for DC-SIGN (SEQ ID NO: 41) and DC-SIGNR (SEQ ID NO: 42) along with the carbohydrate recognition domain (CRD) for DC-SIGN (SEQ ID NO: 39) and DC-SIGNR (SEQ ID NO: 40) are shown.

FIG. 8 shows sequences for CDR1, CDR2 and CDR3 of LIP1 VK and VH dAbs. CDR amino acid sequences correspond to SEQ ID NOs: 43-96. Column 1 from top to bottom are SEQ ID NOs: 45-60. Column 2 from top to bottom are SEQ ID NOs: 61-78. Column 3 from top to bottom are SEQ ID NOs: 79-96.

### DETAILED DESCRIPTION OF THE DISCLOSURE

Within this specification the disclosure has been described, with reference to embodiments, in a way which enables a clear and concise specification to be written. It is intended and should be appreciated that embodiments may be variously combined or separated without parting from the disclosure.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

As used herein, “peptide” refers to about two to about 50 amino acids that are joined together via peptide bonds.

As used herein, “polypeptide” refers to at least about 50 amino acids that are joined together by peptide bonds. Polypeptides generally comprise tertiary structure and fold into functional domains.

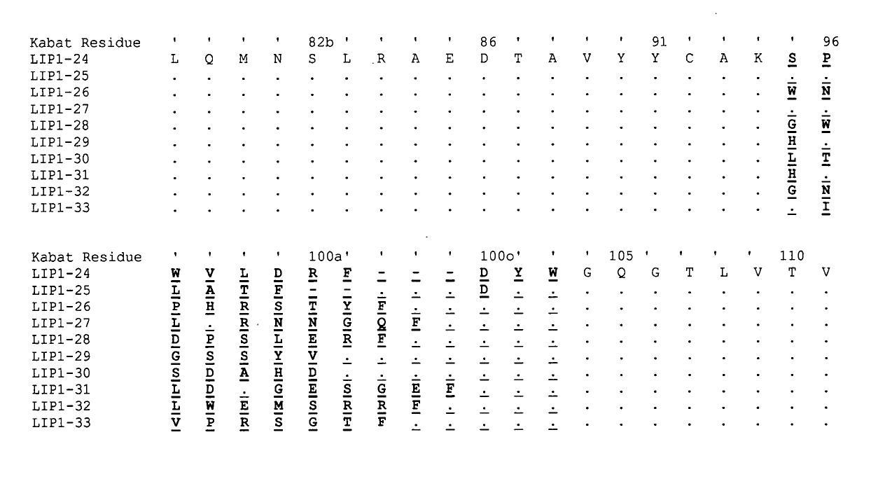

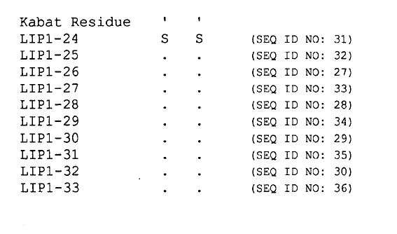

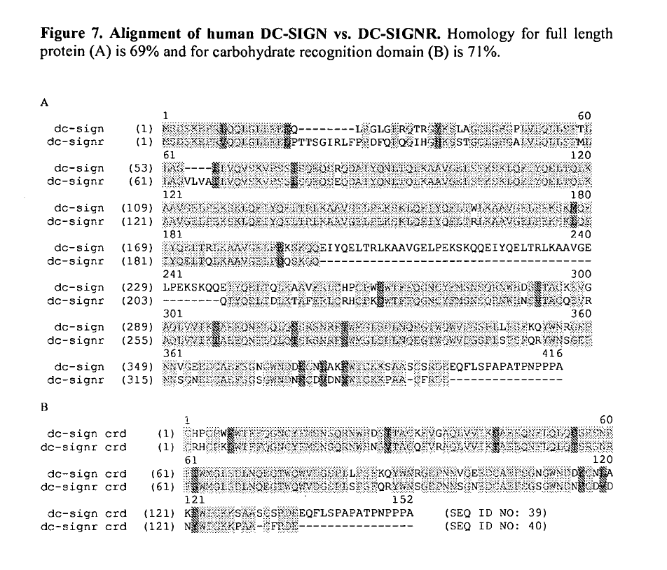

An “isolated” or “purified” polypeptide, antibody or biologically active portion thereof is substantially free of cellular material or other contaminating proteins from the cell or tissue source from which the polypeptide, antibody or biologically active portion thereof is derived, or substantially free from chemical precursors or other chemicals when chemically synthesized. The language “substantially free of cellular material” includes preparations of polypeptide, antibody or biologically active portion thereof in which the protein is separated from cellular components of the cells from which it is isolated or recombinantly produced. In one embodiment, the language “substantially free of cellular material” includes preparations of polypeptide, antibody or biologically active portion thereof having less than about 30% (by dry weight) of non-antibody (also referred to herein as a “contaminating protein”), in one instance, less than about 20% of non-antibody protein, in another instance, less than about 10% of non-antibody protein, and in another instance, less than about 5% non-antibody protein. When the polypeptide, antibody or biologically active portion thereof is purified from a recombinant source, it is also substantially free of culture medium, i.e., culture medium represents less than about 20%, in one instance less than about 10%, and in another instance less than about 5% of the volume of the protein preparation. Dendritic cell-specific ICAM-3 grabbing non-integrin (DC-SIGN or CD209) is a type II membrane protein that is mannose specific calcium dependent (C-type) lectin. DC-SIGN mediates interactions between dendritic cells (DCs) and T cells and is described, for example, by Geijtenbeek et al. Cell (2000); 100, 565-585, Soilleux, Clinical Science (2003), 104, 437-446 with sequence data given in NM_{—}021155 (mRNA) and NP_{—}066978 (protein). The amino acid sequence for human DC-SIGN is also shown in FIG. 7 (SEQ ID NO: 41).

Suitably, the anti-DC-SIGN immunoglobulin single variable domain of the disclosure can be presented in any antibody format.

As used herein an antibody refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format” refers to any suitable polypeptide structure in which one or more anti-DC SIGN antibody single variable domain can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single antibody variable domain (e.g., a dAb, V_{H}, V_{HH}, V_{K}, V_{L}), and modified versions of any of the foregoing (e.g., modified by the covalent attachment of polyethylene glycol or other suitable polymer or a humanized V_{HH}).

Variable domains according to the disclosure may be combined into non-immunoglobulin multi-ligand structures to form multivalent complexes, which bind target molecules with the same antigen, thereby providing superior avidity, while at least one variable domain binds an antigen to increase the half life of the multimer. For example, natural bacterial receptors such as SpA have been used as scaffolds for the grafting of CDRs to generate ligands which bind specifically to one or more epitopes. Details of this procedure are described in U.S. Pat. No. 5,831,012. Other suitable scaffolds include those based on fibronectin and AFFIBODIES™. Details of suitable procedures are described in WO 98/58965. Other suitable scaffolds include lipocallin and CTLA4, as described in van den Beuken et al., J. Mol. Biol. (2001) 310, 591-601, and scaffolds such as those described in WO00/69907 (Medical Research Council), which are based for example on the ring structure of bacterial GroEL or other chaperone polypeptides.

The phrase “immunoglobulin single variable domain” refers to an antibody variable domain (V_{H}, V_{HH}, V_{K}, V_{L}) that specifically binds an antigen or epitope independently of other V regions or domains. The immunoglobulin single variable domains of the disclosure are also described herein as ligands in so far as they are binding ligands for DC-SIGN. An “anti-DC-SIGN” immunoglobulin single variable domain is one which recognizes DC-SIGN or binds specifically to DC-SIGN. In one embodiment, DC-SIGN is human DC-SIGN. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. A “single immunoglobulin variable domain” is the same as an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

Single domain antibodies (single variable domain polypeptides, dAbs) can be generated to have excellent biophysical properties and provide a number of advantages over monoclonal antibodies. For example, dAbs can be generated to be resistant to aggregation, proteolysis and denaturation making them more amenable to the clinical setting. Moreover, their format gives them more flexibility. There can also be significant formatting and manufacturing issues with monoclonal antibodies (which are produced from mammalian expression cells) whereas dAbs can be produced more easily.

A “domain” is a folded protein structure which has tertiary structure independent of the rest of the protein. Generally, domains are responsible for discrete functional properties of proteins, and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

Binding, specific binding and binding affinity of a binding agent such as an antibody or immunoglobulin single variable domain can be determined by measuring the dissociation constant (Kd). Suitable methods for determining Kd include surface plasma resonance. One such method includes the Biacore apparatus available from GE. Other suitable methods include ELISA. See WO2006038027 for details of how to perform competition ELISA and competition BiaCore experiments to determine binding affinity.

In one example, binding is tested using monoclonal phage ELISA. Phage ELISA may be performed according to any suitable procedure. Typically, populations of phage produced at each round of selection for phage expressing binding agents can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope and this can also be undertaken by ELISA using reagents, for example, against a C- or N-terminal tag (see, for example, Winter et al. (1994) Ann. Rev. Immunology 12, 433-55 and references cited therein). In one embodiment, phage ELISA may be performed in the presence of protein L or protein A.

In certain embodiments, the polypeptide, antibody, immunoglobulin single variable domain or dAb specifically binds DC-SIGN, e.g., human DC-SIGN, and dissociates from human DC-SIGN with a dissociation constant (Kd) of 300 nM to 1 pM or 300 nM to 5 pM or 50 nM to 1 pM or 50 nM to 5 pM or 50 nM to 20 pM or about 10 pM or about 15 pM or about 20 pM as determined by surface plasmon resonance. In other embodiments, the polypeptide, antibody, immunoglobulin single variable domain or dAb specifically binds DC-SIGN, e.g., human DC-SIGN, and dissociates from human DC-SIGN with a dissociation constant (Kd) of 400 nM to 1 μM or 500 nM to 1 μM or 600 nM to 1 μM or 700 nM to 1 μM or 800 nM to 1 μM or 900 nM to 1-μM. In other embodiments, the polypeptide, antibody, immunoglobulin single variable domain or dAb specifically binds DC-SIGN, e.g., human DC-SIGN, and dissociates from human DC-SIGN with a dissociation constant (Kd) of 1 to 2 μM or 1 μM to 5 μM or 1 μM to 10 μM or 5 μM to 10 μM or 10 to 20, 30, 40 or 50 μM. In certain embodiments, the polypeptide, antibody, immunoglobulin single variable domain or dAb specifically binds DC-SIGN, e.g., human DC-SIGN, and dissociates from human DC-SIGN with a K_{off} rate constant of 5×10⁻¹ s⁻¹ to 1×10⁻⁷s⁻¹ or 1×10⁻³ s⁻¹ to 1×10⁻⁷ s⁻¹ or 1×10⁻⁴ s⁻¹ to 1×10⁻⁷ s⁻¹ or 1×10⁻⁵ s⁻¹ to 1×10⁻⁷ s⁻¹ or 1×10⁻⁴ s⁻¹ or 1×10⁻⁵ s⁻¹ as determined by surface plasmon resonance. In certain embodiments, the polypeptide, antibody, immunoglobulin single variable domain or dAb specifically binds DC-SIGN, e.g., human DC-SIGN, with a Kₒₙ of 1×10⁻³ M⁻¹s⁻¹ to 1×10⁻⁷ M⁻¹ s⁻¹ or 1×10⁻³ M⁻¹ s⁻¹ to 1×10⁻⁶ M⁻¹s⁻¹ or about 1×10⁻⁴ M⁻¹s⁻¹ or about 1×10⁻⁵ M⁻¹s⁻¹. In one embodiment, the polypeptide, antibody, immunoglobulin single variable domain or dAb specifically binds DC-SIGN, e.g., human DC-SIGN, and dissociates from human DC-SIGN with a dissociation constant (Kd) and a K_{off} as defined in this paragraph. In one embodiment, the polypeptide, antibody, immunoglobulin single variable domain or dAb specifically binds DC-SIGN, e.g., human DC-SIGN, and dissociates from human DC-SIGN with a dissociation constant (Kd) and a Kₒₙ as defined in this paragraph. In some embodiments, the polypeptide, antibody, immunoglobulin single variable domain or dAb specifically binds DC-SIGN (e.g., human DC-SIGN) with a Kd and/or K_{off} and/or Kₒₙ as recited in this paragraph and comprises an amino acid sequence that is at least or at least about 80%, 85%, 90%, 91%, 92%, 93%, 94%, 95%, 96%, 97%, 98%, or 99% identical to the amino acid sequence of LIP1-29. In one embodiment, a “high affinity” binding agent is one which, when in monomeric form, binds a DC-SIGN molecule expressed on a cell surface enabling binding to a cell, such as a dendritic cell.

Typically, a “high affinity” binding agent such as a polypeptide, antibody, immunoglobulin single variable domain or dAb in accordance with the disclosure is one which binds the target molecule, antigen or epitope with a binding affinity (Kd) value of no more than about 300 nM to 1 pM or 300 nM to 5 pM or 50 nM to 1 pM or 50 nM to 5 pM or 50 nM to 20 pM or about 10 pM or about 15 pM or about 20 pM. Suitably, a “low affinity” binding agent in accordance with the present disclosure is one which binds to the target molecule or antigen with a Kd value of 400 nM to 1 μM or 500 nM to 1 μM or 600 nM to 1 μM or 700 nM to 1 μM or 800 nM to 1 μM or 900 nM to 1 μM. In other embodiments, a “low affinity” binding agent in accordance with the disclosure specifically binds DC-SIGN, e.g., human DC-SIGN, and dissociates from human DC-SIGN with a dissociation constant (Kd) of 1 to 2 μM or 1 μM to 5 μM or 1 μM to 10 μM or 5 μM to 10 μM, or 10 to 20, 30, 40 or 50 μM.

In one embodiment, affinity may be determined when the immunoglobulin single variable domains of the disclosure are presented in multivalent phage or crosslinked to protein L.

As described and exemplified herein, dAbs of the disclosure may bind their target DC-SIGN with low affinity. Using a immunoglobulin single variable domain with low affinity can be advantageous.

In particular, a number or plurality of low affinity immunoglobulin single variable domain molecules can be combined in one carrier agent such that a number of interactions between the single variable domain molecules and their cognate binding molecules occur. In this way, the single variable domain molecules can be used to target cells which carry a number or plurality of cognate binding molecules. For example, where a low affinity binding agent, such as an immunoglobulin single variable domain molecule of the disclosure is incorporated onto a carrier molecule in a multiple display format, multiple binding agents should bind to multiple DC-SIGN molecules in order for the carrier agent to bind to or be brought into association with the cell. Such a carrier agent would, advantageously bind those cells with high DC-SIGN expression and not those cells with low DC-SIGN expression. In this way, low affinity binding agents of the disclosure can be used to target specific cells and, in combination, provide overall high affinity binding. In addition, a carrier comprising a plurality of low affinity immunoglobulin single variable domains would have high avidity for cells having a high copy number of DC-SIGN, such as dendritic cells, whilst having only weak avidity to cells having a low copy number of DC-SIGN. Thus, such a carrier would be selective for cells expressing higher levels of DC-SIGN.

Suitable carriers are described, for example, in WO 2007/072022. In one embodiment, the carrier presents a plurality of binding agents in accordance with the disclosure. For example, the carrier may present more than 100 or more than 1000 immunoglobulin single variable domain molecules.

Accordingly, in one aspect of the disclosure there is provided a composition comprising a low affinity dAb in a multiple display format. A multiple display format may include a multimer of immunoglobulin single variable domain molecules in accordance with the disclosure as well as a carrier comprising a plurality of immunoglobulin single variable domain molecules as described above.

In another aspect there is provided a DC-SIGN receptor binding agent comprising an anti-DC-SIGN immunoglobulin single variable domain in accordance with the disclosure. Suitably the binding agent may be a structure comprising one or more anti-DC-SIGN immunoglobulin single variable domains displayed on its surface.

Using low affinity immunoglobulin single variable domain molecules in a multiple display format provides a convenient formulation in which it is not necessary to remove unbound immunoglobulin single variable domain molecules which have not been incorporated onto the carrier from a composition formulation for administration. Free immunoglobulin single variable domain molecules are quickly cleared in vivo. Where these immunoglobulin single variable domain molecules have low affinity for their cognate binding molecule, they are unlikely to bind to receptor and are therefore likely to remain in free circulation and will be cleared.

Calculations of “homology” or “identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In an embodiment, the length of a reference sequence aligned for comparison purposes is at least 30%, or at least 40%, or at least 50%, or at least 60%, or at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences. Amino acid and nucleotide sequence alignments and homology, similarity or identity, as defined herein may be prepared and determined using the algorithm BLAST 2 Sequences, using default parameters (Tatusova, T. A. et al., FEMS Microbiol Lett, 174:187-188 (1999)).

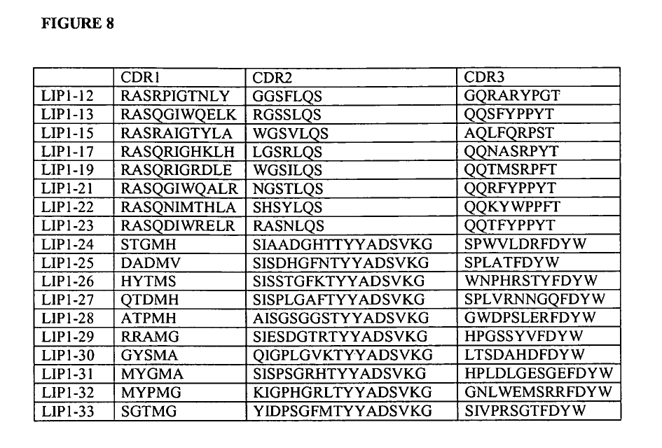

Complementarity Determining Regions (CDRs) and framework regions are those regions of an immunoglobulin variable domain. In particular there are regions of the sequence of a single antibody variable domain which display particular variability i.e. the CDR (complementarity determining region) sequences. The CDRs are at defined positions within the sequence of the antibody variable domain. A number of systems for defining the CDR regions of a sequence will be familiar to those skilled in the art. In one embodiment, the CDR sequences of the present disclosure are as defined in the Kabat database of Sequences of Proteins of Immunological Interest (Kabat E. A., Wu, T. T., Perry, H., Gottesman, K. and Foeller, C. (1991) Sequences of Proteins of Immunological Interest, Fifth Edition. NIH Publication No. 91-3242) which gives a standard numbering scheme for numbering the residues in an antibody in a consistent manner. The immunoglobulin single variable domains (dAbs) described herein contain complementarity determining regions (CDR1, CDR2 and CDR3). In one embodiment, the CDR sequences of the anti-DC SIGN immunoglobulin single variable domains in accordance with the disclosure are those CDRs 1, 2 and 3 as set out in FIG. 8.

The amino acid sequences of the CDRs (CDR1, CDR2, CDR3) of the V_{H} (CDRH1 etc.) and V_{L} (CDRL1 etc.) (V_{K}) dAbs disclosed herein will be readily apparent to the person of skill in the art based on the well known Kabat amino acid numbering system and definition of the CDRs. According to the Kabat numbering system, the most commonly used method based on sequence variability, heavy chain CDR-H3 have varying lengths, insertions are numbered between residue H100 and H101 with letters up to K (i.e. H100, H100A . . . H100K, H101). CDRs can alternatively be determined using the system of Chothia (based on location of the structural loop regions) (Chothia et al., (1989) Conformations of immunoglobulin hypervariable regions; Nature 342 (6252), p877-883), according to AbM (compromise between Kabat and Chothia) or according to the Contact method (based on crystal structures and prediction of contact residues with antigen) as follows. See http://www.bioinf.org.uk/abs/ for suitable methods for determining CDRs.

Once each residue has been numbered, one can then apply the following CDR definitions:

Kabat :

CDR H1: 31-35/35A/35B

CDR H2: 50-65

CDR H3: 95-102

CDR L1: 24-34

CDR L2: 50-56

CDR L3: 89-97

Chothia:

CDR H1: 26-32

CDR H2: 52-56

CDR H3: 95-102

CDR L1: 24-34

CDR L2: 50-56

CDR L3: 89-97

AbM:

| (using Kabat numbering): | (using Chothia numbering): |
|---|---|
| CDR H1: 26-35/35A/35B | 26-35 |
| CDR H2: 50-58 | — |
| CDR H3: 95-102 | — |
| CDR L1: 24-34 | — |
| CDR L2: 50-56 | — |
| CDR L3: 89-97 | — |

Contact

| (using Kabat numbering): | (using Chothia numbering): |
|---|---|
| CDR H1: 30-35/35A/35B | 30-35 |
| CDR H2: 47-58 | — |
| CDR H3: 93-101 | — |
| CDR L1: 30-36 | — |
| CDR L2: 46-55 | — |
| CDR L3: 89-96 | — |

(“—” means the same numbering as Kabat)

The disclosure relates to isolated and/or recombinant nucleic acids encoding peptides or polypeptides described herein.

Nucleic acids referred to herein as “isolated” are nucleic acids which have been separated away from other material (e.g., other nucleic acids such as genomic DNA, cDNA and/or RNA) in its original environment (e.g., in cells or in a mixture of nucleic acids such as a library). An isolated nucleic acid can be isolated as part of a vector (e.g., a plasmid).

Nucleic acids referred to herein as “recombinant” are nucleic acids which have been produced by recombinant DNA methodology, including methods which rely upon artificial recombination, such as cloning into a vector or chromosome using, for example, restriction enzymes, homologous recombination, viruses and the like, and nucleic acids prepared using the polymerase chain reaction (PCR).

The disclosure also relates to a recombinant host cell which comprises a (one or more) recombinant nucleic acid or expression construct comprising a nucleic acid encoding a peptide or polypeptide described herein. There is also provided a method of preparing a peptide or polypeptide, comprising maintaining a recombinant host cell of the disclosure under conditions appropriate for expression of a peptide or polypeptide. The method can further comprise the step of isolating or recovering the peptide or polypeptide, if desired.

For example, a nucleic acid molecule (i.e., one or more nucleic acid molecules) encoding a peptide or polypeptide, or an expression construct (i.e., one or more constructs) comprising such nucleic acid molecule(s), can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g., in the presence of an inducer, in a suitable animal, in suitable culture media supplemented with appropriate salts, growth factors, antibiotics, nutritional supplements, etc.), whereby the encoded peptide or polypeptide is produced. If desired, the encoded peptide or polypeptide can be isolated or recovered (e.g., from the animal, the host cell, medium, milk). This process encompasses expression in a host cell of a transgenic animal (see, e.g., WO 92/03918, GenPharm International). The peptide or polypeptide described herein can also be produced in a suitable in vitro expression system, by chemical synthesis or by any other suitable method. The polypeptide, dAb or antagonist can be expressed in E. coli or in Pichia species (e.g., P. pastoris). In one embodiment, the ligand or dAb monomer is secreted in a quantity of at least about 0.5 mg/L when expressed in E. coli or in Pichia species (e.g., P. pastoris). In one embodiment, the expression vector can be chosen to increase expression into the host cell supernatant. In one embodiment, the expression vector incorporates a GAS leader sequence as described herein. Although, the ligands and dAb monomers described herein can be secretable when expressed in E. coli or in Pichia species (e.g., P. pastoris), they can be produced using any suitable method, such as synthetic chemical methods or biological production methods that do not employ E. coli or Pichia species.

The phrase, “half-life,” refers to the time taken for the serum concentration of the ligand (e.g., dAb, polypeptide or antagonist) to reduce by 50%, in vivo, for example due to degradation of the ligand and/or clearance or sequestration of the ligand by natural mechanisms. The ligands of the disclosure may be stabilized in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. For example, a ligand specific for human serum albumin (HAS) and a target molecule is compared with the same ligand wherein the specificity to HSA is not present, that is does not bind HSA but binds another molecule. For example, it may bind a third target on the cell. Typically, the half-life is increased by 10%, 20%, 30%, 40%, 50% or more. Increases in the range of 2x, 3x, 4x, 5x, 10x, 20x, 30x, 40x, 50x or more of the half-life are possible. Alternatively, or in addition, increases in the range of up to 30x, 40x, 50x, 60x, 70x, 80x, 90x, 100x, 150x of the half-life are possible.

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Half lives (t½ alpha and t½ beta) and AUC can be determined from a curve of serum concentration of ligand against time. The WINNONLIN™ analysis package (available from Pharsight Corp., Mountain View, Calif. 94040, USA) can be used, for example, to model the curve. In a first phase (the alpha phase) the ligand is undergoing mainly distribution in the patient, with some elimination. A second phase (beta phase) is the terminal phase when the ligand has been distributed and the serum concentration is decreasing as the ligand is cleared from the patient. The t alpha half life is the half life of the first phase and the t beta half life is the half life of the second phase. Thus, advantageously, the present disclosure provides a ligand or a composition comprising a ligand according to the disclosure having a tα half-life in the range of 15 minutes or more. In one embodiment, the lower end of the range is 30 minutes, 45 minutes, 1 hour, 2 hours, 3 hours, 4 hours, 5 hours, 6 hours, 7 hours, 10 hours, 11 hours or 12 hours. In addition, or alternatively, a ligand or composition according to the disclosure will have a tα half life in the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours.

Advantageously, the present disclosure provides a ligand or a composition comprising a ligand according to the disclosure having a tβ half-life in the range of 2.5 hours or more. In one embodiment, the lower end of the range is 3 hours, 4 hours, 5 hours, 6 hours, 7 hours, 10 hours, 11 hours, or 12 hours. In addition, or alternatively, a ligand or composition according to the disclosure has a tβ half-life in the range of up to and including 21 days. In one embodiment, the upper end of the range is 12 hours, 24 hours, 2 days, 3 days, 5 days, 10 days, 15 days or 20 days. Advantageously a ligand or composition according to the disclosure will have a tβ half life in the range 12 to 60 hours. In a further embodiment, it will be in the range 12 to 48 hours. In a further embodiment still, it will be in the range 12 to 26 hours.

In addition, or alternatively to the above criteria, the present disclosure provides a ligand or a composition comprising a ligand according to the disclosure having an

AUC value (area under the curve) in the range of 1 mg/min/ml or more. In one embodiment, the lower end of the range is 5, 10, 15, 20, 30, 100, 200 or 300 mg/min/ml. In addition, or alternatively, a ligand or composition according to the disclosure has an AUC in the range of up to 600 mg/min/ml. In one embodiment, the upper end of the range is 500, 400, 300, 200, 150, 100, 75 or 50 mg/min/ml.

Advantageously, a ligand according to the disclosure will have a AUC in the range selected from, but preferably not limited to, the group consisting of the following: 15 to 150 mg/min/ml, 15 to 100 mg/min/ml, 15 to 75 mg/min/ml, and 15 to 50 mg/min/ml.

In one embodiment, a (one or more) half-life extending moiety (e.g., albumin, transferrin and fragments and analogues thereof) is conjugated or associated with the anti-DC-SIGN immunoglobulin single variable domain or dAb of the disclosure. Examples of suitable albumin, albumin fragments or albumin variants for use in an anti-DC-SIGN immunoglobulin single variable domain-binding format are described in WO 2005077042, which disclosure is incorporated herein by reference and forms part of the disclosure of the present text.

Further examples of suitable albumin, fragments and analogs for use in anti-DC-SIGN immunoglobulin single variable domain-binding format are described in WO 03076567, which disclosure is incorporated herein by reference and which forms part of the disclosure of the present text.

Where a (one or more) half-life extending moiety (e.g., albumin, transferrin and fragments and analogues thereof) or other fusion protein is used to format the anti-DC-SIGN immunoglobulin single variable domain polypeptides and dAbs of the disclosure, it can be conjugated using any suitable method, such as, by direct fusion to the anti-DC-SIGN immunoglobulin single variable domain (e.g., dAb), for example by using a single nucleotide construct that encodes a fusion protein, wherein the fusion protein is encoded as a single polypeptide chain with the half-life extending moiety located N- or C-terminally to the anti-DC-SIGN immunoglobulin single variable domain. Alternatively, conjugation can be achieved by using a peptide linker between moieties, e.g., a peptide linker as described in WO 03076567 or WO 2004003019 (these linker disclosures being incorporated by reference in the present disclosure to provide examples for use in the present disclosure). Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

In embodiments of the disclosure described throughout this disclosure, instead of the use of an anti-DC-SIGN immunoglobulin single variable domain “dAb” of the disclosure, it is contemplated that the skilled addressee can use a polypeptide or domain that comprises one or more or all 3 of the CDRs of a dAb of the disclosure that binds DC-SIGN (e.g., CDRs grafted onto a suitable protein scaffold or skeleton, e.g. an affibody, an SpA scaffold, an LDL receptor class A domain or an EGF domain). The disclosure as a whole is to be construed accordingly to provide disclosure of anti-DC-SIGN immunoglobulin single variable domain polypeptides using such domains in place of a dAb. In this respect, see WO2008/096158.

Generally, the anti-DC-SIGN immunoglobulin single variable domain, polypeptide, ligand or binding agent of the disclosure will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates. In one embodiment, the anti-DC-SIGN immunoglobulin single variable domain of the disclosure may be arrayed onto a vesicle such as a micelle or liposome.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The anti-DC-SIGN immunoglobulin single variable domain, polypeptide, ligand or binding agent of the present disclosure may be used as separately administered compositions or in conjunction with other agents. Pharmaceutical compositions can include “cocktails” of various cytotoxic or other agents in conjunction with the ligands of the present disclosure, or even combinations of ligands according to the present disclosure having different specificities, such as ligands selected using different target antigens or epitopes, whether or not they are pooled prior to administration.

The route of administration of pharmaceutical compositions according to the disclosure may be any of those commonly known to those of ordinary skill in the art. For therapy, including without limitation immunotherapy, the selected immunoglobulin single variable domains thereof of the disclosure can be administered to any patient in accordance with standard techniques.

The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician. Administration can be local (e.g., local delivery to the lung by pulmonary administration, e.g., intranasal administration) or systemic as indicated.

The immunoglobulin single variable domains, polypeptides, ligands or binding agents of this disclosure can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate.

The compositions containing the present immunoglobulin single variable domains, polypeptides, ligands or binding agents or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of immunoglobulin single variable domain, e.g. dAb or antagonist per kilogram of body weight, with doses of 0.05 to 2.0 mg/kg/dose being more commonly used. For prophylactic applications, compositions containing the present immunoglobulin single variable domains or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease.

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale. A sustained (e.g., one day or more, or longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing an immunoglobulin single variable domain, polypeptide, ligand or binding agent or cocktail thereof according to the present disclosure may be utilised in prophylactic and therapeutic settings to aid in the alteration, inactivation, killing or removal of a select target cell population in a mammal. The composition may also block infection by blocking the receptor which normally mediates entry of an infectious agent such as HIV, Hepatitis C or Ebola virus. In addition, the selected repertoires of polypeptides described herein may be used extracorporeally or in vitro selectively to kill, deplete or otherwise effectively remove a target cell population or an infectious agent from a heterogeneous collection of cells. Blood from a mammal may be combined extracorporeally with the ligands whereby the undesired cells or infectious agents are killed or otherwise removed from the blood for return to the mammal in accordance with standard techniques.

A composition containing a ligand (e.g., antagonist) according to the present disclosure may be utilised in prophylactic and therapeutic settings to aid in the alteration, inactivation, killing or removal of a select target cell population in a mammal.

The immunoglobulin single variable domains, polypeptides, ligands or binding agents can be administered and or formulated together with one or more additional therapeutic or active agents. When a immunoglobulin single variable domain (e.g., a dAb) is administered with an additional therapeutic agent, the ligand can be administered before, simultaneously with or subsequent to administration of the additional agent. Generally, the ligand and additional agent are administered in a manner that provides an overlap of therapeutic effect.

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds target antigen) administered to a subject over the course of one day (24 hours) (daily dose), two days, one week, two weeks, three weeks or one or more months (e.g., by a single administration, or by two or more administrations). The interval between doses can be any desired amount of time.

The disclosure is further described in the following examples, for the purposes of illustration only.

### EXAMPLES

### Example 1

### Lead selection and characterisation of domain antibodies to DC-SIGN.

Domain antibodies generated were derived from 4G and 6G phage libraries. The 4G libraries are based on a single human framework for VH (V3-23 [locus] DP47 [V Base Entry] and JH4b) and VL (012/02 [locus] DPK9 [V Base Entry] and Jk1) with side chain diversity incorporated at positions in the antigen binding site that make contacts with antigen in known molecular structures (WO2005093074). Importantly, these positions are also highly diverse in the mature repertoire. The canonical structure (VH:

1-3, Vk: 2-1-1) encoded by these frameworks are by far the most common in the human antibody repertoire. The CDR3 of the heavy chain was designed to be as short as possible yet still able to form an antigen binding surface. The libraries can be selected and affinity matured without knowing the sequence of selected clones.

The 6G libraries are based on a single human framework for VH (V3-23 [locus] DP47 [V Base Entry] and JH4b) and VL (012/02 [locus] DPK9 [V Base Entry] and Jk1) with side chain diversity incorporated at positions in the antigen binding site that make contacts with antigen in known molecular structures (see WO04101790).

The 6G dAb libraries incorporate additional diversification to improve the folding efficiency of the 4G library. In the VH and Vk sequences only a few amino acids are critical for folding efficiency. These are located in the H1 loop of VH DP-47 and at the boundary of framework 2/CDR2 in Vk DPk9. In Library 6G, diversification was targeted to these regions to improve the likelihood of selecting dAbs with improved folding. Tyrosines at position 32 and 49 were diversified in the VH and Vk scaffold, respectively. In the Vk scaffold, residues 27 and 89 were also diversified to create a continuous and larger diversified surface. Improved folding was selected for by a heat treatment of the primary phage library before clean up on protein-A or -L. Libraries were then created by recombining pooled CDR1+2 library fragments, derived from primary libraries, with a library of pooled CDR3 fragments.

For selections, human DC-SIGN or a DC-SIGN peptide corresponding to a 9×HIS tag, a linker and the C-terminal end of DC-SIGN. (Amino acid sequence: HHHHHHHHH-SGSG-KKSAASCSRDEEQFLSPAPATPNPPPA (SEQ ID NO: 37)), was coated to Maxisorp immunotubes (Nunc) (5-50 μg/ml in PBS or 0.1 M NaHCO₃ buffer, pH 9.6). Typically 50 μg/ml antigen is used in the first round of selection with 10¹² TU of phage in 1 ml PBSM. In subsequent rounds the amount of antigen is reduced in each round. The phage/antigen mixture is incubated for 1 hour. The immunotubes beads are washed eight times with PBST and eight times with PBS. Bound phage were eluted in 0.5 ml of 100 μg/m1 trypsin in PBS during 10 min, then used to infect 2 ml of log-phase E. coli TG1 cells at 37° C. during 30 min. Serial dilutions (for phage titer) and library plating were performed on 2xTY-Tet agar plates. For the next selection round, cells were scraped from the plates and used to inoculate 200 ml of 2xTY-Tet at 37° C. for phage amplification. Supernatant were used for phage preparation and bacterial cell pellets were used to isolate phage dsDNA for subcloning of pooled dAb genes into the bacterial expression vector pDOM5 (see below).

For phage ELISA, the ELISA wells were coated overnight at 4° C. with DC SIGN or DC SIGNR (R&D Cat nr 162-D2) at (1-10 μg/ml in PBS or 0.1 M NaHCO₃ buffer, pH 9.6). After blocking the wells with PBS containing 2% skimmed-milk powder (PBSM), phage was incubated in PBSM for 1 hr. After washing with PBS, bound phage were detected using a conjugate of horseradish peroxidase with an anti-M13 monoclonal antibody (Amersham) using 3,3′,5,5′-tetramethylbenzidine as substrate.

Specific phage positives that recognize DC SIGN but did not recognise DC SIGNR were obtained after two and three rounds of selection. The selected dAb genes were subcloned from the pDOM4 phage vector into pDOM5. (pDOM4, as described in WO 2007/085815, is a derivative of the Fd phage vector in which the gene III signal peptide sequence is replaced with the yeast glycolipid anchored surface protein (GAS) signal peptide (WO 2005/093074). It also contains a c-myc tag between the leader sequence and gene III, which puts the gene III back in frame).

pDOM5 is a pUC 119-based expression vector under control of the LacZ promoter. Expression of dAbs into the supernatant was ensured by fusion to the universal GAS leader signal peptide at the N-terminal end (described, for example in WO 2005/093074). The dAbs are preceded by Ser-Thr residues which are present in the polylinker to accommodate a SalI cloning site. In addition, a c-myc-tag was appended at the C-terminal end of the dAbs. After transformation of E. coli HB2151 cells, colonies were used to inoculate 50 to 500 mL of Terrific Broth medium supplemented with carbenicillin (100 μg per mL). Induction was performed with the OVERNIGHT EXPRESS™ SYSTEM 1 (high-level protein expression system, Novagen) according to the manufacturer's instructions. The cultures were incubated at 30° C. for 24-48 hours with shaking at 250 rpm. After cell pelleting by centrifugation (4,000 rpm for 20 min), the supernatants were filtered using a 0.45 μm filter and incubated overnight at 4° C. with STREAMLINE™-protein A beads (Amersham Biosciences, binding capacity: 5 mg of dAb per mL of beads) for the V_{H} dAbs, or Protein L-sepharose beads (Affitech, binding capacity: 2 mg of dAb per mL of beads) for the V_{L} dAbs. The beads were then packed into drip columns, washed with 10 column volumes of PBS, and bound dAbs were eluted in 0.1 M glycine-HCl, pH 2.0 or 3.0 for the V_{H} and V_{L} dAbs, respectively. After neutralisation with 1 M Tris-HCl, pH 8.0, the protein samples were dialyzed in PBS and concentrated on VIVASPIN™ 5-kDa concentrators (Vivascience) before storage at 4° C. Protein purity was estimated by visual analysis after SDS-PAGE on 12% acrylamide Tris-glycine gel (Invitrogen). Protein concentrations and yields (in mg per L of bacterial culture) were measured at 280 nm, using extinction coefficients calculated from the amino acid compositions.

For ELISA assays with soluble dAbs, the antigens were coated as described in the phage ELISA protocol. The wells were blocked with PBS containing 2% TWEEN™ (PBST) and dAbs were incubated in PBST for 1 hr. After washing with PBS, bound dAbs were detected with mAb 9E10 (Sigma, 1/2000 dilution) followed by rabbit anti-mouse conjugated with horseradish peroxidase (Sigma, 1/2000 dilution). This ELISA was also performed whereby the dAbs were incubated in the presence of protein L (1 ug/ml).

The dAbs did not yield any positive ELISA signals when tested as soluble dAbs. It is likely that the affinities as monomeric dAbs were too low.

In the case of protein L cross-linking, 129 binding clones were identified. These were sequenced and re-tested for binding in ELISA to DC-SIGN and DC-SIGNR. 40 unique clones were identified. Some clones bound DC-SIGN specifically (and not DC-SIGNR) in the presence of protein L.

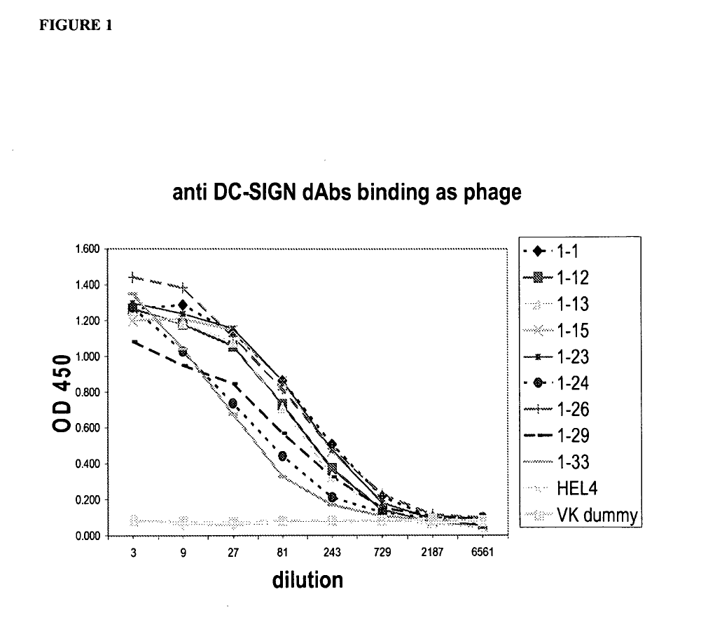

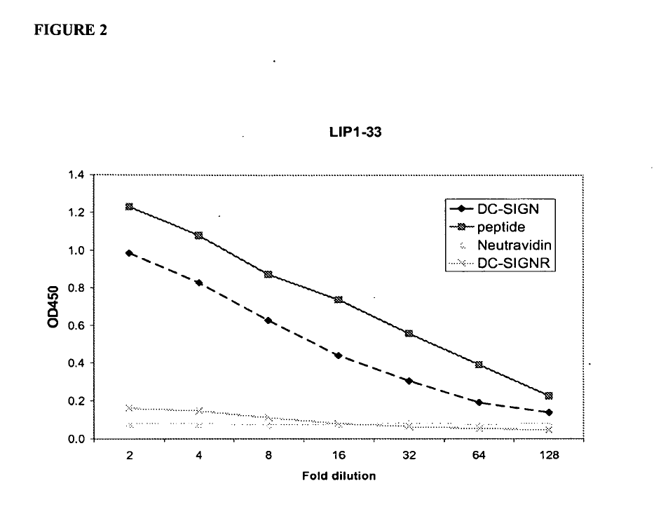

Several clones were identified that bound DC SIGN specifically (and did not bind DC SIGNR) as phage but give undetectable binding as soluble dAbs. This yielded several VH clones (see FIG. 1).

Several selections approaches were performed to identify clones that bind only DC-SIGN and the DC-SIGN peptide (unique C-terminal end of DC-SIGN).

Selection approaches:

1. Round 1, Round 2 and Round 3 with DC-SIGN protein.

2. Round 1 and Round 2 with DC-SIGN protein and Round 3 on the DC-SIGN peptide

3. Round 1 and Round 3 with DC-SIGN protein and Round 2 on the DC-SIGN peptide

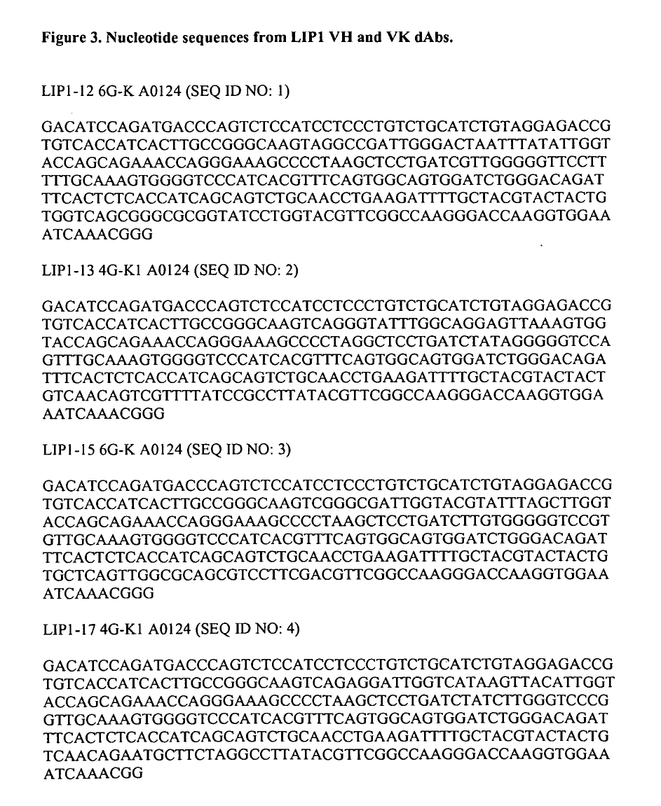

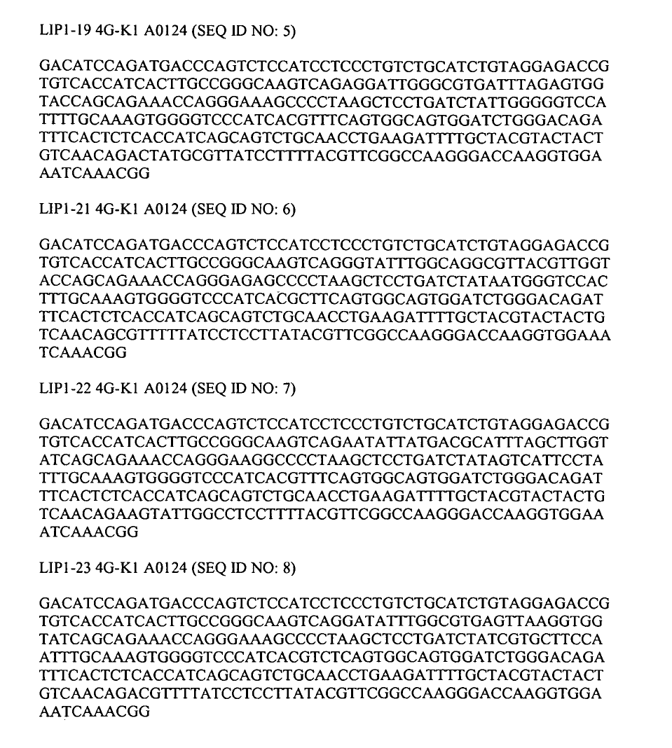

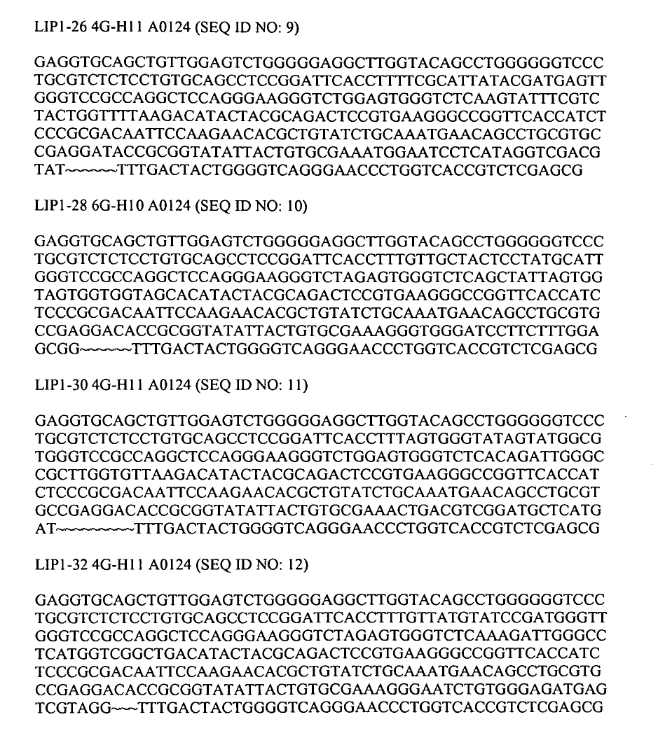

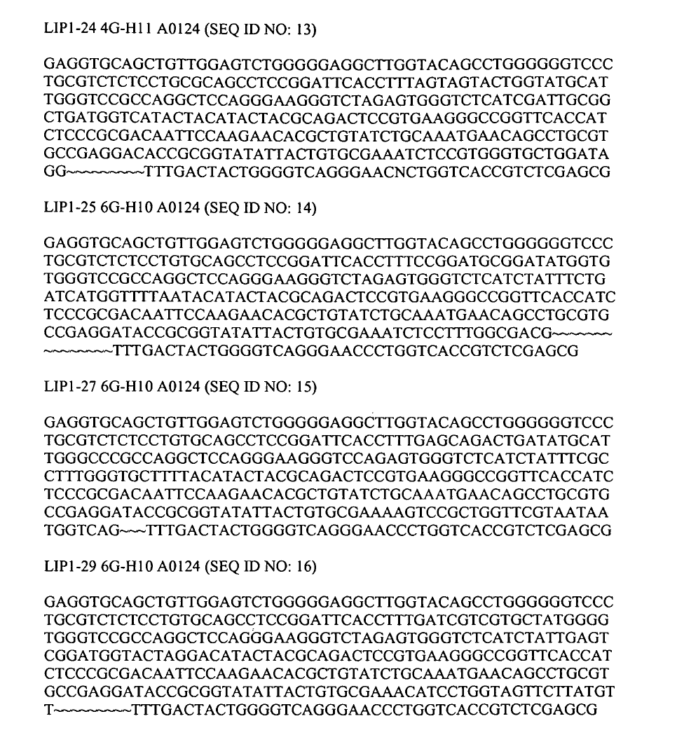

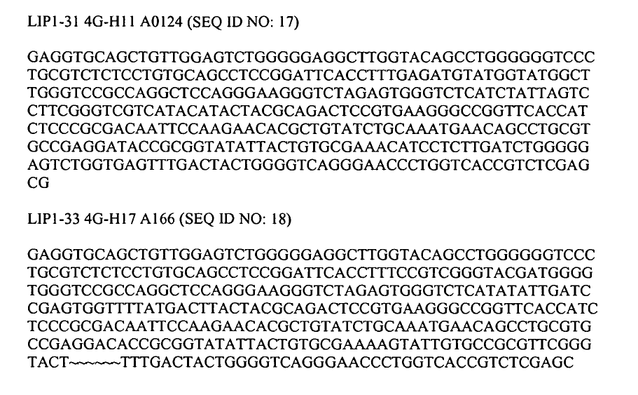

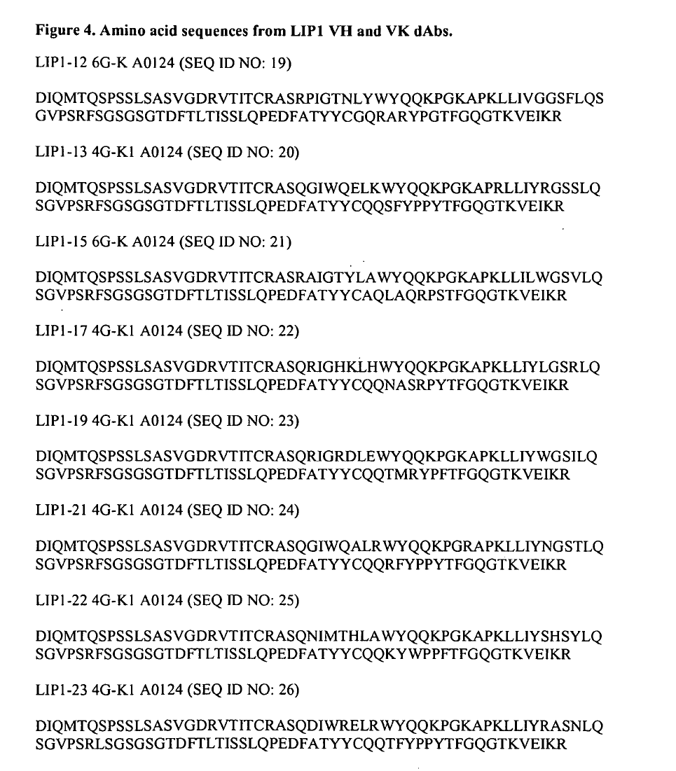

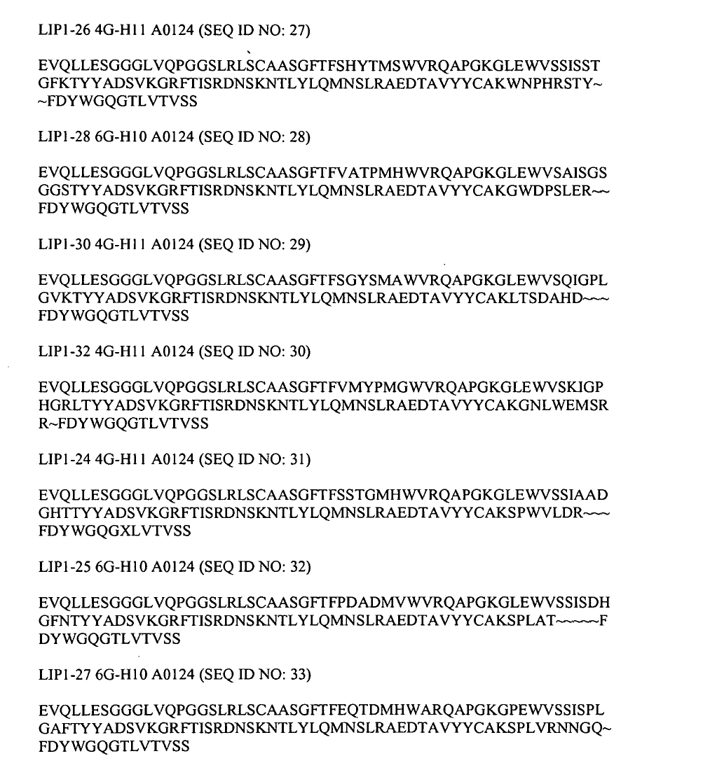

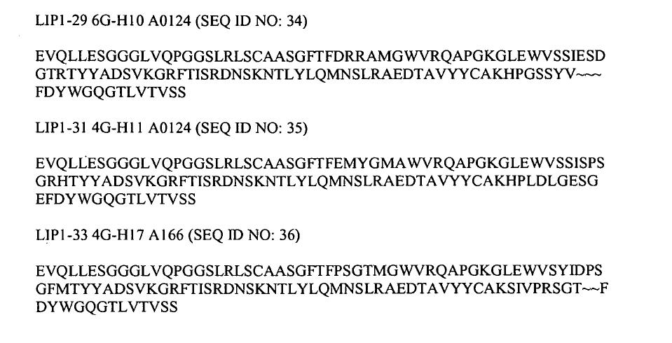

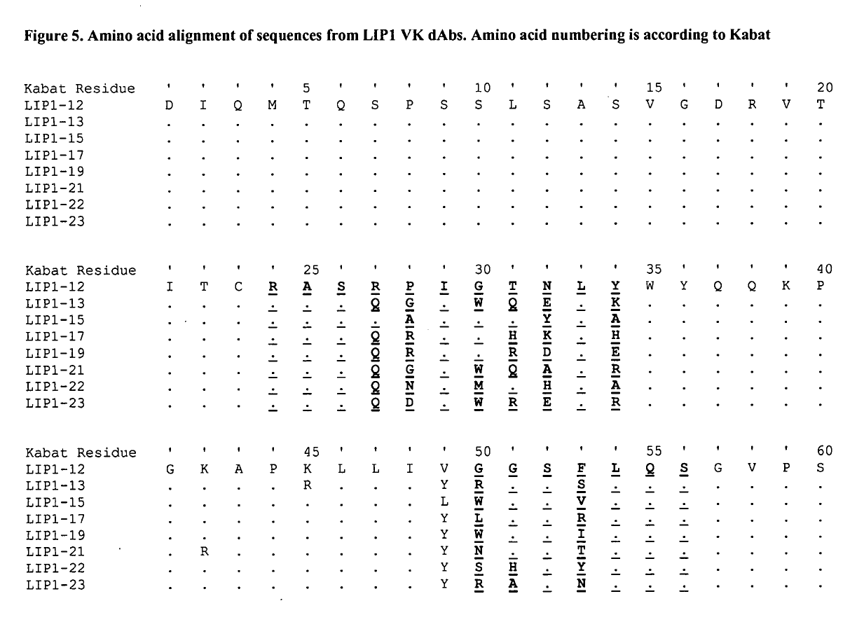

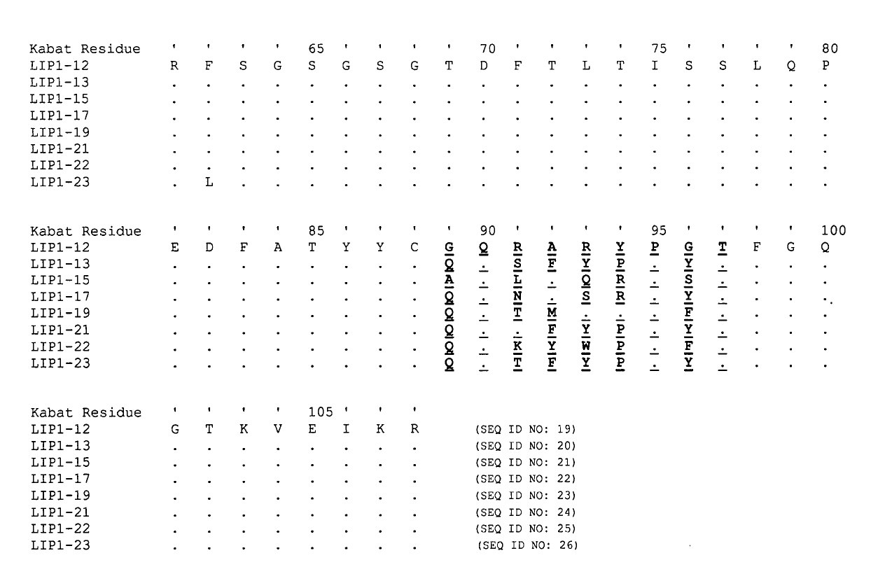

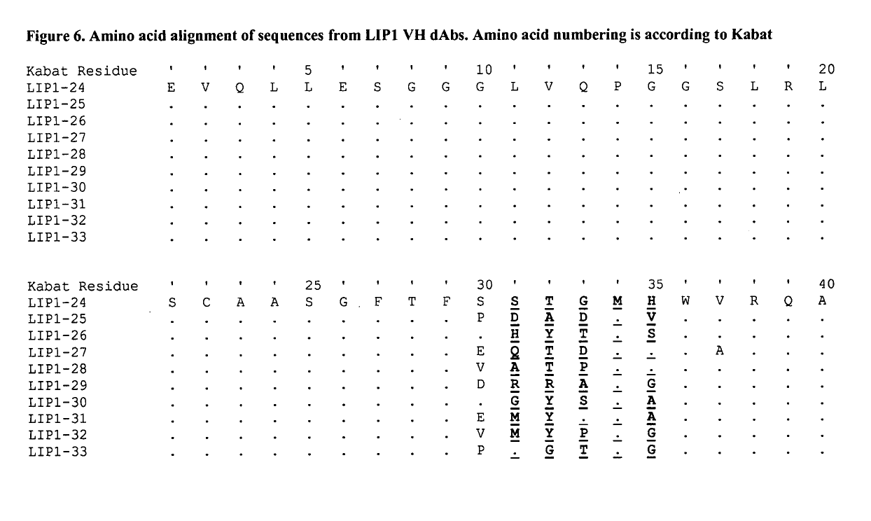

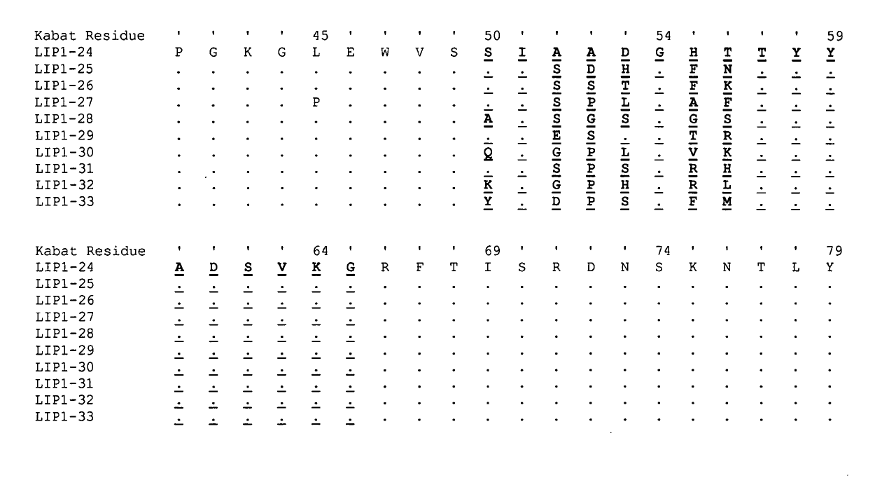

In total 2200 clones from the three approaches were screened in phage ELISA. 1000 clones were screened from the selection with the DC-SIGN peptide (R1 and R3 with DC-SIGN protein and R2 on the peptide). The nucleotide and amino acid sequences of selected clones are set in FIGS. 3 and 4. Out of seven phage positives from the primary screen only one clone (LIP1-33) was found to bind specifically to DC-SIGN and DC-SIGN peptide. It was also found that LIP1-33 binding to the DC-SIGN peptide can be inhibited by DC-SIGN and both HIS tagged and biotinylated DC-SIGN peptide but not with control proteins (data not shown). LIP1-33 and other DC-SIGN specific phage VH clones from the DC SIGN selections were recloned into pDOM5 in which a GHHGHHGHHGHHGHH tag (SEQ ID NO: 38) was appended at the C-terminal end. Soluble dAbs were expressed and purified. VH dAb HEL4 (Jespers et al., J. Mol. Biol. (2004) 337, 893-903) was included as a negative control.

TABLE 1
dAbs with GHHGHHGHHGHHGHH tag (10xHIS) (SEQ ID NO: 38)

| | | | Expression |
|---|---|---|---|
| Name | dAb type | pI | mg/L |
| LIP1-25 | VH | 6.60 | 0.5 |
| LIP1-27 | VH | 7.34 | 0.6 |
| LIP1-28 | VH | 7.34 | 0.4 |
| LIP1-29 | VH | 8.04 | 1.4 |
| LIP1-30 | VH | 7.33 | 0.5 |
| LIP1-31 | VH | 6.66 | 0.2 |
| LIP1-33 | VH | 8.04 | 1.5 |
| HEL4 | VH | 6.36 | 0.7 |

While this disclosure has been particularly shown and described with references to embodiments thereof, it will be understood by those skilled in the art that various changes in form and details may be made therein without departing from the scope of the disclosure encompassed by the appended claims.

1. An anti-DC-SIGN immunoglobulin single variable domain.

2. The anti-DC-SIGN immunoglobulin single variable domain according to claim 1, wherein the immunoglobulin single variable domain binds to human DC-SIGN with a dissociation constant (K_{d}) of 1-50 μM, as determined by surface plasmon resonance.

3. An isolated polypeptide comprising an amino acid sequence that is at least 70% identical to at least one amino acid sequence selected from the group consisting of: SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) and SEQ ID NO: 36 (LIP1-33) and which binds to human DC-SIGN.

4. An isolated polypeptide comprising an amino acid sequence selected from the group consisting of: SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) and SEQ ID NO: 36 (LIP1-33).

5. An isolated polypeptide encoded by a nucleotide sequence that is at least 60% identical to the nucleotide sequence selected from the group consisting of: SEQ ID NO: 1 (LIP1-12), SEQ ID NO: 2 (LIP1-13), SEQ ID NO: 3 (LIP1-15), SEQ ID NO: 4 (LIP1-17), SEQ ID NO: 5 (LIP1-19), SEQ ID NO: 6 (LIP1-21), SEQ ID NO: 7 (LIP1-22), SEQ ID NO: 8 (LIP1-23), SEQ ID NO: 9 (LIP1-26), SEQ ID NO: 10 (LIP1-28), SEQ ID NO: 11 (LIP1-30), SEQ ID NO: 12 (LIP1-32), SEQ ID NO: 13 (LIP1-24), SEQ ID NO: 14 (LIP1-25), SEQ ID NO: 15 (LIP1-27), SEQ ID NO: 16 (LIP1-29), SEQ ID NO: 17 (LIP1-31) and SEQ ID NO: 18 (LIP1-33) and which binds to human DC-SIGN.

6. An anti-DC-SIGN immunoglobulin single variable domain comprising an amino acid sequence that is at least 90% identical to the amino acid sequence of any one amino acid sequence selected from the group consisting of: SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) and SEQ ID NO: 36 (LIP1-33) and which binds to human DC-SIGN.

7. An anti-DC-SIGN immunoglobulin single variable domain comprising an amino acid sequence that is identical to the amino acid sequence of SEQ ID NO: 32 (LIP1-29), SEQ ID NO: 33 (LIP1-30), or SEQ ID NO: 36 (LIP1-33).

8. An anti-DC-SIGN immunoglobulin single variable domain comprising an amino acid sequence selected from the group consisting of: SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) and SEQ ID NO: 36 (LIP1-33).

9. An anti-DC-SIGN immunoglobulin single variable domain comprising an amino acid sequence of any one of the amino acid sequences set out in SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) that is modified at no more than 25 amino acid positions and comprises a CDR1 sequence that is at least 50% identical to the CDR1 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

10. An anti-DC-SIGN immunoglobulin single variable domain comprising an amino acid sequence of any one of the amino acid sequences set out in SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) that is modified at no more than 25 amino acid positions and comprises a CDR2 sequence that is at least 50% identical to the CDR2 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

11. An anti-DC-SIGN immunoglobulin single variable domain comprising an amino acid sequence of any one of the amino acid sequences set out in SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) that is modified at no more than 25 amino acid positions and comprises a CDR3 sequence that is at least 50% identical to the CDR3 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

12. An anti-DC-SIGN immunoglobulin single variable domain comprising an amino acid sequence of any one of the amino acid sequences set out in SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) that is modified at no more than 25 amino acid positions and comprises a CDR1 sequence that is at least 50% identical to a CDR1 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) and comprises a CDR2 sequence that is at least 50% identical to a CDR2 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

13. An anti DC SIGN anti-DC-SIGN immunoglobulin single variable domain comprising an amino acid sequence of any one of the amino acid sequences set out in SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) that is modified at no more than 25 amino acid positions and comprises a CDR1 sequence that is at least 50% identical to the CDR1 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) and comprises a CDR3 sequence that is at least 50% identical to the CDR3 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

14. An anti DC SIGN anti-DC-SIGN immunoglobulin single variable domain comprising an amino acid sequence of any one of the amino acid sequences set out in SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) that is modified at no more than 25 amino acid positions and comprises a CDR2 sequence that is at least 50% identical to the CDR2 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) and comprises a CDR3 sequence that is at least 50% identical to the CDR3 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

15. An anti DC SIGN anti-DC-SIGN immunoglobulin single variable domain comprising an amino acid sequence of any one of the amino acid sequences set out in SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) that is modified at no more than 25 amino acid positions and comprises a CDR1 sequence that is at least 50% identical to the CDR1 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) and comprises a CDR2 sequence that is at least 50% identical to the CDR2 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33) and comprises a CDR3 sequence that is at least 50% identical to the CDR3 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

16. An anti-DC-SIGN immunoglobulin single variable domain comprising a CDR3 sequence that is at least 50% identical to a CDR3 sequence selected from the group consisting of: the CDR3 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

17. An anti-DC-SIGN immunoglobulin single variable domain comprising a CDR3 sequence selected from the group consisting of: the CDR3 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

18. An anti-DC-SIGN immunoglobulin single variable domain comprising at least one CDR selected from the group consisting of: CDR1, CDR2, and CDR3, wherein the CDR1, CDR2, or CDR3 is identical to a CDR1, CDR2, or CDR3 sequence in any one of SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) or SEQ ID NO: 36 (LIP1-33).

19. An anti-DC-SIGN immunoglobulin single variable domain as claimed in claim 1 which binds to DC-SIGN with low affinity.

20. A ligand that has binding specificity for DC-SIGN and inhibits the binding of an anti-DC-SIGN immunoglobulin single variable domain comprising an amino acid sequence selected from the group consisting of: SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) and SEQ ID NO: 36 (LIP1-33) .

21. An isolated polypeptide encoded by a nucleotide sequence that is at least 80% identical to a nucleotide sequence selected from the group consisting of: SEQ ID NO: 1 (LIP1-12), SEQ ID NO: 2 (LIP1-13), SEQ ID NO: 3 (LIP1-15), SEQ ID NO: 4 (LIP1-17), SEQ ID NO: 5 (LIP1-19), SEQ ID NO: 6 (LIP1-21), SEQ ID NO: 7 (LIP1-22), SEQ ID NO: 8 (LIP1-23), SEQ ID NO: 9 (LIP1-26), SEQ ID NO: 10 (LIP1-28), SEQ ID NO: 11 (LIP1-30), SEQ ID NO: 12 (LIP1-32), SEQ ID NO: 13 (LIP1-24), SEQ ID NO: 14 (LIP1-25), SEQ ID NO: 15 (LIP1-27), SEQ ID NO: 16 (LIP1-29), SEQ ID NO: 17 (LIP1-31) and SEQ ID NO: 18 (LIP1-33) and wherein the polypeptide comprises an amino acid sequence that is at least 90% identical to an amino acid sequence selected from the group consisting of: SEQ ID NO: 19 (LIP1-12), SEQ ID NO: 20 (LIP1-13), SEQ ID NO: 21 (LIP1-15), SEQ ID NO: 22 (LIP1-17), SEQ ID NO: 23 (LIP1-19), SEQ ID NO: 24 (LIP1-21), SEQ ID NO: 25 (LIP1-22), SEQ ID NO: 26 (LIP1-23), SEQ ID NO: 27 (LIP1-26), SEQ ID NO: 28 (LIP1-28), SEQ ID NO: 29 (LIP1-30), SEQ ID NO: 30 (LIP1-32), SEQ ID NO: 31 (LIP1-24), SEQ ID NO: 32 (LIP1-25), SEQ ID NO: 33 (LIP1-27), SEQ ID NO: 34 (LIP1-29), SEQ ID NO: 35 (LIP1-31) and SEQ ID NO: 36 (LIP1-33).

22. An anti-DC-SIGN immunoglobulin single variable domain or peptide as claimed in claim 1 which binds specifically to DC-SIGN but not to DC-SIGNR.

23. An isolated or recombinant nucleic acid encoding a polypeptide comprising an anti-DC-SIGN immunoglobulin single variable domain as claimed in claim 1.

24. A vector comprising a nucleic acid as claimed in claim 23.

25. A host cell comprising a nucleic acid as claimed in claim 23.

26. A method of producing a polypeptide comprising an anti-DC-SIGN immunoglobulin single variable domain, the method comprising maintaining a host cell as claimed in claim 25 under conditions suitable for expression of said nucleic acid or vector, whereby a polypeptide comprising an immunoglobulin single variable domain is produced.

27. An anti-DC-SIGN immunoglobulin single variable domain which immunoglobulin single variable domain has the binding specificity of any one of LIP1-12, LIP1-13, LIP1-15, LIP1-17, LIP1-19, LIP1-21, LIP1-22, LIP1-23, LIP1-24, LIP1-25, LIP1-26, LIP1-27, LIP1-28, LIP1-29, LIP1-30, LIP1-31, LIP1-32 or LIP1-33.

28. An anti-DC-SIGN immunoglobulin single variable domain as claimed in claim 3 wherein the amino acid sequence further comprises amino acids ST at the N-terminal.

29. An anti-DC-SIGN immunoglobulin single variable domain as claimed in any of claim 3 wherein the amino acid sequence further comprises a His-tag at the C-terminal end.
